(19) United States
(12) Patent Application Publication
(10) Pub. No.: US 2025/0250252 A1
(43) Pub. Date: Aug. 7, 2025

(54) PIPERIDINYLBENZONITRILE DERIVATIVES AS INHIBITORS OF GLUTAMINYL-PEPTIDE CYCLOTRANSFERASE AND GLUTAMINYL-PEPTIDE CYCLOTRANSFERASE LIKE PROTEIN

(71) Applicant: Boehringer Ingelheim International GmbH, Ingelheim am Rhein (DE)

(72) Inventors: Jens WILLWACHER, Biberach an der Riss (DE); Georg DAHMANN, Biberach (DE); Sandra Ruth HANDSCHUH, Biberach an der Riss (DE); Christian Andreas KUTTRUFF, Donaueschingen (DE); Sophia Astrid REINDL, Mittelbiberach (DE)

(21) Appl. No.: 19/041,049

(22) Filed: Jan. 30, 2025

(30) Foreign Application Priority Data
Feb. 6, 2024 (EP) 24156131.5

(51) Int. Cl. C07D 401/14; A61K 31/4545; A61K 31/501; A61K 31/506; A61K 45/06; C07D 413/14

(52) U.S. Cl. CPC C07D 401/14; A61K 31/4545; A61K 31/501; A61K 31/506; A61K 45/06; C07D 413/14

(57) ABSTRACT

The present disclosure provides certain piperidinylphenylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### RELATED APPLICATION DISCLOSURE

This application claims the benefit under 35 U.S.C. § 119 of EP Application No. 24156131.5 filed Feb. 6, 2024, of which is hereby incorporated by reference herein in its entirety.

### TECHNICAL FIELD

The present disclosure provides certain piperidinylphenylcarbonitrile derivatives, and pharmaceutically acceptable salts thereof, that are inhibitors of Glutaminyl-peptide cyclotransferase (QPCT) and glutaminyl-peptide cyclotransferase-like protein (QPCTL), and are therefore useful for the treatment of diseases treatable by inhibition of QPCT/L. Also provided are pharmaceutical compositions containing the same, and processes for preparing said compounds.

### BACKGROUND INFORMATION

Glutaminyl-peptide cyclotransferase (QPCT) and glutaminyl-peptide cyclotransferase-like protein (QPCTL) catalyze the intramolecular cyclization of N-terminal glutamine (Q) residues into pyroglutamic acid (pE) liberating ammonia [Stephan Schilling et al., “Identification of Human Glutaminyl Cyclase as a Metalloenzyme POTENT INHIBITION BY IMIDAZOLE DERIVATIVES AND HETEROCYCLIC CHELATORS,” Journal of Biological Chemistry 278, no. 50 (2003): 49773-79, https://doi.org/10.1074/jbc.m309077200; Holger Cynis et al., “Isolation of an Isoenzyme of Human Glutaminyl Cyclase: Retention in the Golgi Complex Suggests Involvement in the Protein Maturation Machinery,” Journal of Molecular Biology 379, no. 5 (2008): 966-80, https://doi.org/10.1016/j.jmb.2008.03.078; Anett Stephan et al., “Mammalian Glutaminyl Cyclases and Their Isoenzymes Have Identical Enzymatic Characteristics,” FEBS Journal 276, no. 22 (2009): 6522-36, 30 https://doi.org/10.1111/j.1742-4658.2009.07337.x.]. While QPCT is a secreted protein, QPCTL is retained within the Golgi complex. Both enzymes share a high homology in the active site and similar catalytic specificity. Because of the high homology in the active site, inhibition of the active site blocks the enzymatic activity of both enzymes: QPCT and QPCTL. Hence the term “QPCT/L” describes both enzymes at once. Due to their different cellular localisation, differences in their relevance for modification of biological substrates have been reported. Known substrates of the intracellular QPCTL and/or extracellular QPCT are CD47 [Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.], different chemokines (like for example CCL2 and 7 or CX3CL1) [Rosa Barreira da Silva et al., “Loss of the Intracellular Enzyme QPCTL Limits Chemokine Function and Reshapes Myeloid Infiltration to Augment Tumor Immunity,” Nature Immunology 23, no. 4 (2022): 568-80, https://doi.org/10.1038/s41590-022-01153-x; Astrid Kehlen et al., “N-Terminal Pyroglutamate Formation in CX3CL1 Is Essential for Its Full Biologic Activity,” Bioscience Reports 37, no. 4 (2017): BSR20170712, https://doi.org/10.1042/bsr20170712.], Amyloid-b peptides [Cynis et al., “Isolation of an Isoenzyme of Human Glutaminyl Cyclase: Retention in the Golgi Complex Suggests Involvement in the Protein Maturation Machinery.” ] or hormones like TRH [Andreas Becker et al., “IsoQC (QPCTL) Knock-out Mice Suggest Differential Substrate Conversion by Glutaminyl Cyclase Isoenzymes,” Biological Chemistry 397, no. 1 (2016): 45-55, https://doi.org/10.1515/hsz-2015-0192.]. The modification of N-terminal glutamine to pyroglutamate on the substrates has functional consequences for the proteins and could impact different pathomechanisms in several diseases. CD47 is expressed on the cell surface of virtually all cells of the body, including apoptotic cells, senescent cells or cancer cells. [Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011]. The main ligand for CD47 is signal-regulatory protein alpha (SIRPα), an inhibitory transmembrane receptor present on myeloid cells, such as macrophages, monocytes, neutrophils, dendritic cells and others. QPCTL mediated N-terminal pyroglutamate modification on CD47 is required for SIRPα binding [Deborah Hatherley et al., “Paired Receptor Specificity Explained by Structures of Signal Regulatory Proteins Alone and Complexed with CD47,” Molecular Cell 31, no. 2 (2008): 266-77, https://doi.org/10.1016/j.molcel.2008.05.026; Meike E. W. Logtenberg et al., “Glutaminyl Cyclase Is an Enzymatic Modifier of the CD47-SIRPα Axis and a Target for Cancer Immunotherapy,” Nature Medicine 25, no. 4 (2019): 612-19, https://doi.org/10.1038/s41591-019-0356-z.] This signaling axis induces a “Don't Eat Me Signal”, preventing engulfment of CD47 expressing cells by macrophages. Thus, high expression of CD47 is connected to the pathogenesis of cancer [Logtenberg et al., “Glutaminyl Cyclase Is an Enzymatic Modifier of the CD47-SIRPα Axis and a Target for Cancer Immunotherapy,” 2019; Meike E. W. Logtenberg, Ferenc A. Scheeren, and Ton N. Schumacher, “The CD47-SIRPα Immune Checkpoint,” Immunity 52, no. 5 (2020): 742-52, https://doi.org/10.1016/j.immuni.2020.04.011.], COVID-19 [Katie-May McLaughlin et al., “A Potential Role of the CD47/SIRPalpha Axis in COVID-19 Pathogenesis,” Current Issues in Molecular Biology 43, no. 3 (2021): 1212-25, https://doi.org/10.3390/cimb43030086.], lung fibrosis [Gerlinde Wernig et al., “Unifying Mechanism for Different Fibrotic Diseases,” Proceedings of the National Academy of Sciences 114, no. 18 (2017): 4757-62, https://doi.org/10.1073/pnas.1621375114; Lu Cui et al., “Activation of JUN in Fibroblasts Promotes Pro-Fibrotic Programme and Modulates Protective Immunity,” Nature Communications 11, no. 1 (2020): 2795, https://doi.org/10.1038/s41467-020-16466-4.], systemic sclerosis [Wernig et al., “Unifying Mechanism for Different Fibrotic Diseases”; Tristan Lerbs et al., “CD47 Prevents the Elimination of Diseased Fibroblasts in Scleroderma,” JCI Insight 5, no. 16 (2020): e140458, https://doi.org/10.1172/jci.insight.140458.] and liver fibrosis [Taesik Gwag et al., “AntiCD47 Antibody Treatment Attenuates Liver Inflammation and Fibrosis in Experimental Non-alcoholic Steatohepatitis Models,” Liver International 42, no. 4 (2022): 829-41, https://doi.org/10.1111/liv.15182.]. Since enhanced CD47 expression blocks the clearance of apoptotic cells, there is an accrual of apoptotic lung epithelial cells, leading to a profibrotic stimulus and accelerating lung inflammation and -scaring [Alexandra L. McCubbrey and Jeffrey L. Curtis, “Efferocytosis and Lung Disease,” Chest 143, no. 6 (2013): 1750-57, https://doi.org/10.1378/chest.12-2413; Brennan D. Gerlach et al., “Efferocytosis Induces Macrophage Proliferation to Help Resolve Tissue Injury,” Cell Metabolism, 2021, https://doi.org/10.1016/j.cmet.2021.10.015.]. Since CD47 half-life and function is majorly dependent on QPCTL enzyme activity, QPCT and QPCTL inhibition could be a suitable mechanism as a treatment in lung fibrosis such as IPF or SSC-ILD [Lerbs et al., “CD47 Prevents the Elimination of Diseased Fibroblasts in Scleroderma.” ], alone or together with current standard of care in pulmonary fibrosis like Nintedanib [Luca Richeldi et al., “Efficacy and Safety of Nintedanib in Idiopathic Pulmonary Fibrosis,” The New England Journal of Medicine 370, no. 22 (2014): 2071-82, https://doi.org/10.1056/nejmoa1402584; Kevin R Flaherty et al., “Nintedanib in Progressive Fibrosing Interstitial Lung Diseases,” New England Journal of Medicine 381, no. 18 (2019): 1718-27, https://doi.org/10.1056/nejmoal908681.] or future treatments like a PDE4 inhibitor [Luca Richeldi et al., “Trial of a Preferential Phosphodiesterase 4B Inhibitor for Idiopathic Pulmonary Fibrosis,” New England Journal of Medicine 386, no. 23 (2022): 2178-87, https://doi.org/10.1056/nejmoa2201737].

By expression of CD47, cancer cells can evade destruction by the immune system or evade immune surveillance, e.g. by evading phagocytosis by immune cells [Stephen B. Willingham et al., “The CD47-Signal Regulatory Protein Alpha (SIRPa) Interaction Is a Therapeutic Target for Human Solid Tumors,” Proceedings of the National Academy of Sciences 109, no. 17 (2012): 6662-67, https://doi.org/10.1073/pnas.1121623109].

In addition to CD47, chemokines, such as CCL2 and CX3CL1, have been identified as QPCTL and/or QPCT substrates [Holger Cynis et al., “The Isoenzyme of Glutaminyl Cyclase Is an Important Regulator of Monocyte Infiltration under Inflammatory Conditions,” EMBO Molecular Medicine 3, no. 9 (2011): 545-58, https://doi.org/10.1002/emmm.201100158]. The formation of the N-terminal pGlu was shown to increase in vivo activity, both by conferring resistance to aminopeptidases and by increasing its capacity to induce chemokine receptor signaling. Two main monocyte chemoattractants CCL2 and CCL7 are insensitive to DPP4-inactivation in vivo because of an intracellular mechanism of N-terminal cyclization mediated by the Golgi-associated enzyme QPCTL. It has been shown that QPCTL is a critical regulator of monocyte migration into solid tumors [Kaspar Bresser et al., “QPCTL Regulates Macrophage and Monocyte Abundance and Inflammatory Signatures in the Tumor Microenvironment,” Oncoimmunology 11, no. 1 (2022): 2049486, https://doi.org/10.1080/2162402x.2022.2049486; Rosa Barreira da Silva et al., “Loss of the Intracellular Enzyme QPCTL Limits Chemokine Function and Reshapes Myeloid Infiltration to Augment Tumor Immunity,” Nature Immunology, 2022, 1-13, https://doi.org/10.1038/s41590-022-01153-x]. Targeting of chemokines has long been pursued as a potential strategy for modulating cellular trafficking in different disease settings.

It is therefore desirable to provide potent QPCT/L inhibitors.

WO 2023/205173 discloses QPCTL modulators of the general formula:

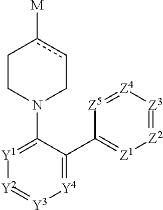

which includes compound 14:

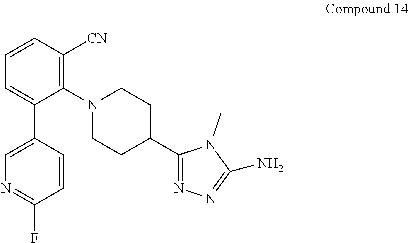 [SMILES: Cn1c(N)nnc1C1CCN(c2c(C#N)cccc2-c2ccc(F)nc2)CC1]

Compound 14 in WO 2023/205173 is disclosed therein [00470] as having inhibitory activity on isolated QPCTL of IC₅₀<1 μM and cellular activity in A549 cells of EC₅₀<1 μM.

Yu, L., Zhao, P., Sun, Y. et al. Sig Transduct Target Ther 8, 454 (2023) (herein “STTT 2023”) disclose compounds QP5020 and QP5038 as potent benzonitrile-based inhibitors of glutaminyl-peptide cyclotransferase-like protein (QPCTL) with antitumor efficacy:

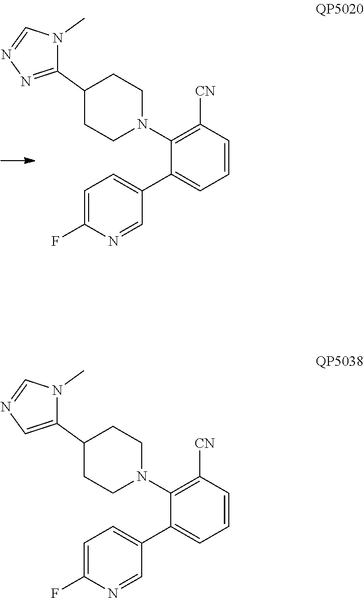 [SMILES: Cn1cnnc1C1CCN(c2c(C#N)cccc2-c2ccc(F)nc2)CC1 ; Cn1cncc1C1CCN(c2c(C#N)cccc2-c2ccc(F)nc2)CC1]

Compound QP5020 is disclosed therein as having QPCTL inhibition activity of IC₅₀ 15.0+/−5.5 nM and QP5038 as having QPCTL inhibition activity of IC₅₀ 3.8+/−0.7 nM.

WO 2024/020517 discloses inhibitors of general formula:

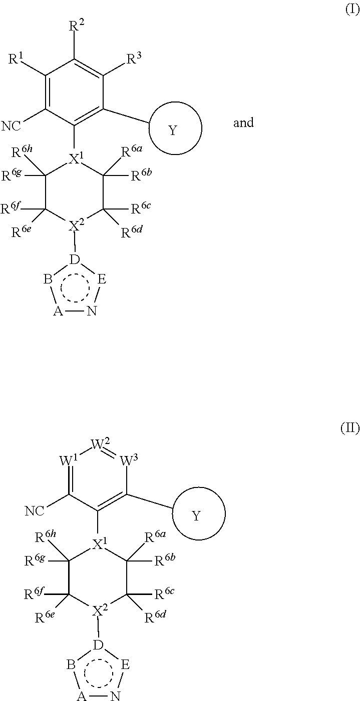

which includes compound (1):

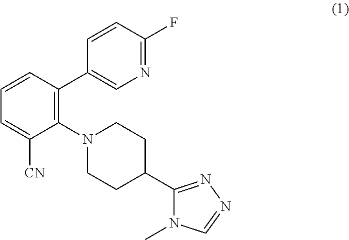 [SMILES: Cn1cnnc1C1CCN(c2c(C#N)cccc2-c2ccc(F)nc2)CC1]

Compound (1) in WO 2024/020517 is disclosed therein [00698] as having inhibitory activity on isolated QPCTL of IC₅₀<0.1 μM, cellular activity in Ramos cells of IC₅₀<0.1 μM, and inhibitory activity of DLD-1 cellular QPCTL activity in an imaging assay of <0.1 μM. Compound (1) in WO 2024/020517 and QP5020 are identical.

Further selected examples in WO 2024/020517 are:

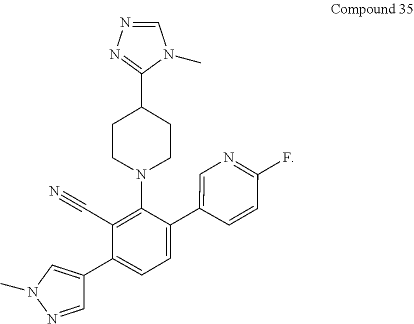 [SMILES: Cn1cc(-c2ccc(-c3ccc(F)nc3)c(N3CCC(c4nncn4C)CC3)c2C#N)cn1]

Inhibitory activity on isolated QPCTL of IC₅₀<0.1 μM;
Inhibitory activity of DLD-1 cellular QPCTL activity in an imaging assay of <0.1 μM.

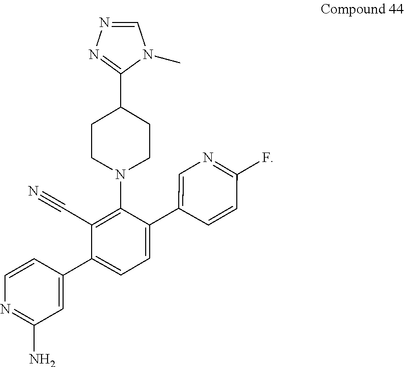 [SMILES: Cn1cnnc1C1CCN(c2c(-c3ccc(F)nc3)ccc(-c3ccnc(N)c3)c2C#N)CC1]

Inhibitory activity on isolated QPCTL of IC₅₀<0.1 μM;
Inhibitory activity of DLD-1 cellular QPCTL activity in an imaging assay of <0.1 μM.

### BRIEF DESCRIPTION OF THE DRAWINGS

FIG. 1 shows the increase in permeability in CACO-2 cells between compared pairs of compounds. FIG. 1 shows that introduction of the fluoro substituent on the piperidyl ring at the 4-position leads to increased permeability for QPCT/L inhibitors bearing a pyrido- or benzonitrile core.

FIG. 2 shows the decrease in efflux between the compared pairs of compounds. FIG. 2 shows that introduction of the fluoro substituent on the piperidyl ring at the 4-position leads to decreased efflux ratio for QPCT/L inhibitors bearing a pyrido- or benzonitrile core.

### DETAILED DESCRIPTION OF THE INVENTION

The present invention discloses novel piperidinylphenylcarbonitrile derivatives of formula (I)

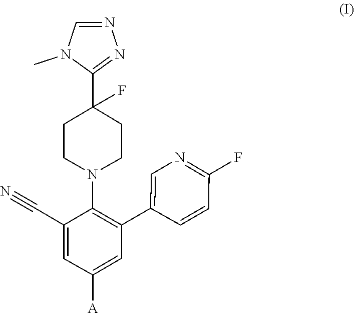

that are inhibitors of Glutaminyl-peptide cyclotransferase (QPCT) and glutaminyl-peptide cyclotransferase-like protein (QPCTL), possessing appropriate pharmacological and pharmacokinetic properties enabling their use as medicaments for the treatment of conditions and/or diseases treatable by inhibition of QPCT/L.

The compounds of the present invention may provide several advantages, such as enhanced potency, cellular potency, high metabolic and/or chemical stability, high selectivity, safety and tolerability, enhanced solubility, enhanced permeability, desirable plasma protein binding, enhanced bioavailability, suitable pharmacokinetic profiles, and the possibility to form stable salts.

### Compounds of the Invention

The present invention provides novel piperidinylphenylcarbonitrile derivatives that surprisingly, are potent inhibitors of QPCT and QPCTL (Assay A), as well as potent inhibitors of QPCT/L in cells relevant for, but not limited to, lung diseases or cancer, (Assay B).

Furthermore, the present novel piperidinylphenylcarbonitrile derivatives have appropriate membrane permeability and a low in vitro efflux (Assay C).

Furthermore, the compounds of the present invention have a favorable CYP induction profile as indicated by a low n-fold induction of CYP3A4 mRNA after incubation with the compound at 10 μM concentration (Assay D).

Furthermore, the compounds of the present invention show improved stability in murine hepatocytes that facilitates preclinical compound evaluation (Assay E).

Compounds of the present invention bear a fluoro substituent attached to the 4-position of the piperidyl ring (noted herein below as “4-fluoropiperidyl” in the tables), which show surprisingly higher permeability in CACO2-Cells and reduced efflux, (Assay C). This effect is demonstrated with the following comparisons with the analogous non-fluoro compound:

| Compound without 4-fluoropiperidyl | Compound with 4-fluoropiperidyl |
|---|---|
| 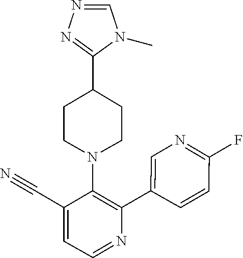 [SMILES: Cn1cnnc1C1CCN(c2c(C#N)ccnc2-c2ccc(F)nc2)CC1] Example 133 in WO 2024/020517 CACO2 Perm. 4.8 x 10⁻⁶ cm/sec Efflux ratio (BA/AB) 3.5 | 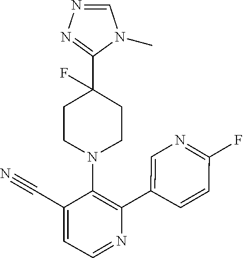 [SMILES: Cn1cnnc1C1(F)CCN(c2c(C#N)ccnc2-c2ccc(F)nc2)CC1] Example 1 in EP 22188580.9 CACO2 Perm. 28.0 x 10⁻⁶ cm/sec Efflux ratio (BA/AB) 2.3 |
| 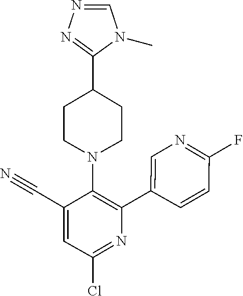 [SMILES: Cn1cnnc1C1CCN(c2c(C#N)cc(Cl)nc2-c2ccc(F)nc2)CC1] Example 18 in WO 2024/020517 CACO2 Perm. 23.0 x 10⁻⁶ cm/sec Efflux ratio (BA/AB) 3.3 | 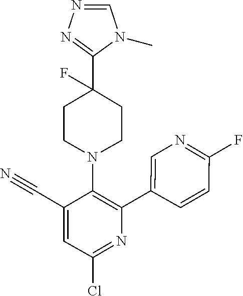 [SMILES: Cn1cnnc1C1(F)CCN(c2c(C#N)cc(Cl)nc2-c2ccc(F)nc2)CC1] Example 18 in EP 23161417.3 CACO2 Perm. 66.0 x 10⁻⁶ cm/sec Efflux ratio (BA/AB) 1.0 |
| 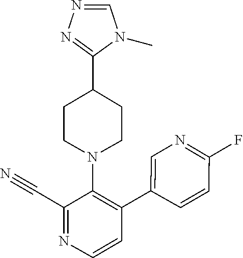 [SMILES: Cn1cnnc1C1CCN(c2c(-c3ccc(F)nc3)ccnc2C#N)CC1] Example 132 in WO 2024/020517 CACO2 Perm. 1.8 x 10⁻⁶ cm/sec Efflux ratio (BA/AB) 7.8 | 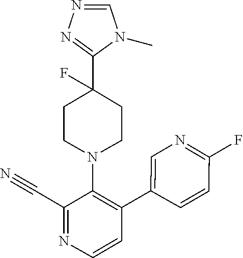 [SMILES: Cn1cnnc1C1(F)CCN(c2c(-c3ccc(F)nc3)ccnc2C#N)CC1] Example 10 in EP 22188580.9 CACO2 Perm. 11.0 x 10⁻⁶ cm/sec Efflux ratio (BA/AB) 5.6 |
| 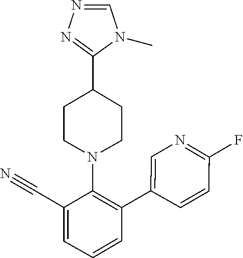 [SMILES: Cn1cnnc1C1CCN(c2c(C#N)cccc2-c2ccc(F)nc2)CC1] QP5020 / Example 1 in WO 2024/020517 CACO2 Perm. 28.0 x 10⁻⁶ cm/sec Efflux ratio (BA/AB) 2.4 | 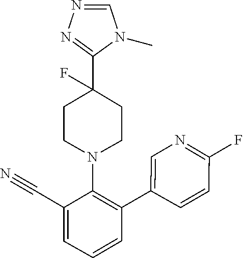 [SMILES: Cn1cnnc1C1(F)CCN(c2c(C#N)cccc2-c2ccc(F)nc2)CC1] Example 56 in EP 23161417.3 CACO2 Perm. 50.0 x 10⁻⁶ cm/sec Efflux ratio (BA/AB) 0.7 |
| 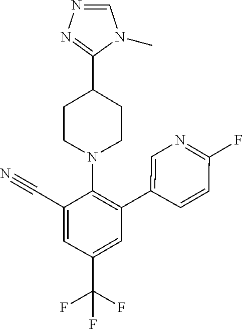 [SMILES: Cn1cnnc1C1CCN(c2c(C#N)cc(C(F)(F)F)cc2-c2ccc(F)nc2)CC1] Example 70 in WO 2024/020517 CACO2 Perm. 9.0 x 10⁻⁶ cm/sec Efflux ratio (BA/AB) 10.4 | 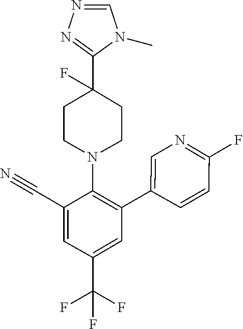 [SMILES: Cn1cnnc1C1(F)CCN(c2c(C#N)cc(C(F)(F)F)cc2-c2ccc(F)nc2)CC1] Example 4 in EP 23161417.3 CACO2 Perm. 40.5 x 10⁻⁶ cm/sec Efflux ratio (BA/AB) 1.3 |
| 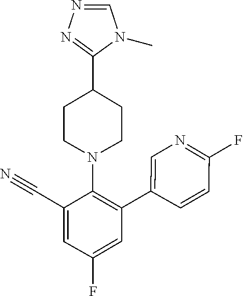 [SMILES: Cn1cnnc1C1CCN(c2c(C#N)cc(F)cc2-c2ccc(F)nc2)CC1] Example 130 in WO 2024/020517 CACO2 Perm. 24.0 x 10⁻⁶ cm/sec Efflux ratio (BA/AB) 2.1 | 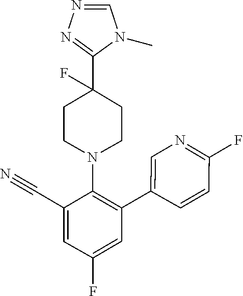 [SMILES: Cn1cnnc1C1(F)CCN(c2c(C#N)cc(F)cc2-c2ccc(F)nc2)CC1] Example 6 in EP 23161417.3 CACO2 Perm. 44.0 x 10⁻⁶ cm/sec Efflux ratio (BA/AB) 1.6 |
| 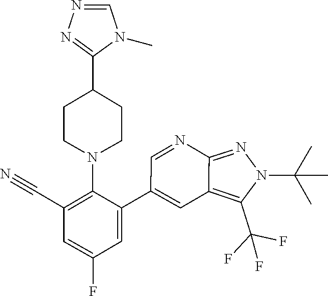 [SMILES: Cn1cnnc1C1CCN(c2c(C#N)cc(F)cc2-c2cnc3nn(C(C)(C)C)c(C(F)(F)F)c3c2)CC1] Hitherto unpublished reference compound CACO2 Perm. 17.0 x 10⁻⁶ cm/sec Efflux ratio (BA/AB) 5.7 | 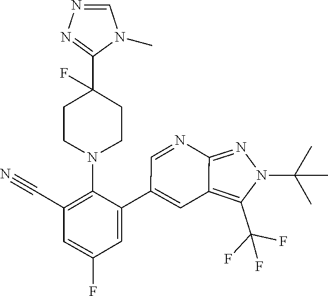 [SMILES: Cn1cnnc1C1(F)CCN(c2c(C#N)cc(F)cc2-c2cnc3nn(C(C)(C)C)c(C(F)(F)F)c3c2)CC1] Hitherto unpublished reference compound CACO2 Perm. 51.0 x 10⁻⁶ cm/sec Efflux ratio (BA/AB) 0.8 |
| 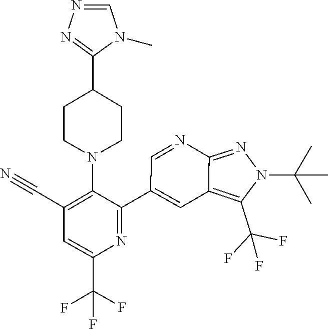 [SMILES: Cn1cnnc1C1CCN(c2c(C#N)cc(C(F)(F)F)nc2-c2cnc3nn(C(C)(C)C)c(C(F)(F)F)c3c2)CC1] Example 13 in EP 23189886.7 CACO2 Perm. 3.7 x 10⁻⁶ cm/sec Efflux ratio (BA/AB) 5.7 | 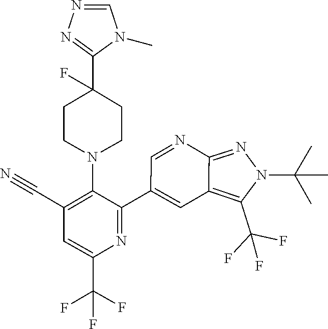 [SMILES: Cn1cnnc1C1(F)CCN(c2c(C#N)cc(C(F)(F)F)nc2-c2cnc3nn(C(C)(C)C)c(C(F)(F)F)c3c2)CC1] Example 4 in EP 23189886.7 CACO2 Perm. 99.5 x 10⁻⁶ cm/sec Efflux ratio (BA/AB) 0.6 |
| 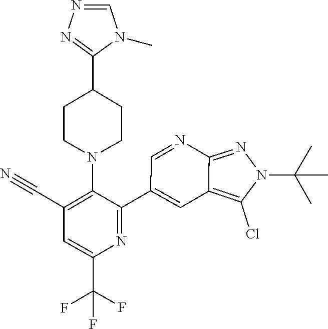 [SMILES: Cn1cnnc1C1CCN(c2c(C#N)cc(C(F)(F)F)nc2-c2cnc3nn(C(C)(C)C)c(Cl)c3c2)CC1] Hitherto unpublished reference compound CACO2 Perm. 1.0 x 10⁻⁶ cm/sec Efflux ratio (BA/AB) 36.4 | 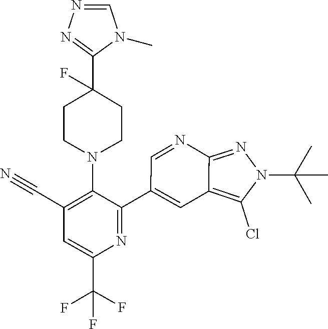 [SMILES: Cn1cnnc1C1(F)CCN(c2c(C#N)cc(C(F)(F)F)nc2-c2cnc3nn(C(C)(C)C)c(Cl)c3c2)CC1] Example 3 in EP 23189886.7 CACO2 Perm. 47.0 x 10⁻⁶ cm/sec Efflux ratio (BA/AB) 1.0 |
| 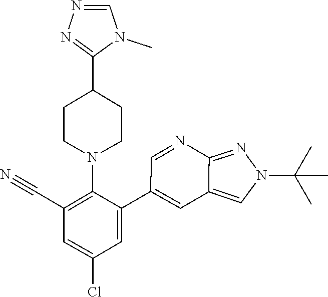 [SMILES: Cn1cnnc1C1CCN(c2c(C#N)cc(Cl)cc2-c2cnc3nn(C(C)(C)C)cc3c2)CC1] Example 17 in EP 23161417.3 CACO2 Perm. 2.5 x 10⁻⁶ cm/sec Efflux ratio (BA/AB) 21.2 | 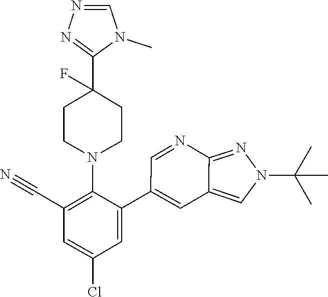 [SMILES: Cn1cnnc1C1(F)CCN(c2c(C#N)cc(Cl)cc2-c2cnc3nn(C(C)(C)C)cc3c2)CC1] Example 19 in EP 23161417.3 CACO2 Perm. 18.0 x 10⁻⁶ cm/sec Efflux ratio (BA/AB) 4.5 |
| 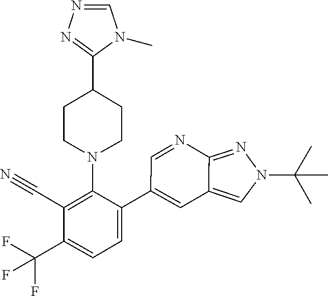 [SMILES: Cn1cnnc1C1CCN(c2c(-c3cnc4nn(C(C)(C)C)cc4c3)ccc(C(F)(F)F)c2C#N)CC1] Example 32 in EP 23161417.3 CACO2 Perm. 0.2 x 10⁻⁶ cm/sec Efflux ratio (BA/AB) 145.8 | 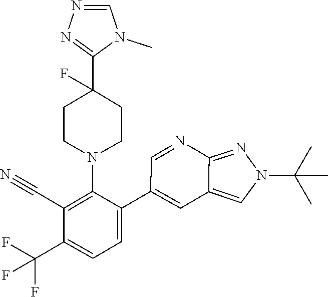 [SMILES: Cn1cnnc1C1(F)CCN(c2c(-c3cnc4nn(C(C)(C)C)cc4c3)ccc(C(F)(F)F)c2C#N)CC1] Example 30 in EP 23161417.3 CACO2 Perm. 1.7 x 10⁻⁶ cm/sec Efflux ratio (BA/AB) 22.9 |
| 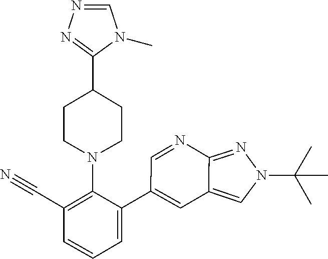 [SMILES: Cn1cnnc1C1CCN(c2c(C#N)cccc2-c2cnc3nn(C(C)(C)C)cc3c2)CC1] Example 50 in EP 22216126.7 CACO2 Perm. 0.5 x 10⁻⁶ cm/sec Efflux ratio (BA/AB) 75.5 | 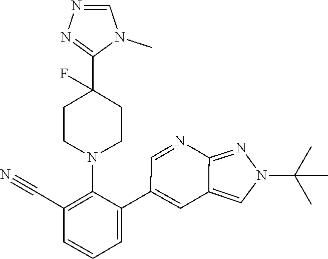 [SMILES: Cn1cnnc1C1(F)CCN(c2c(C#N)cccc2-c2cnc3nn(C(C)(C)C)cc3c2)CC1] Example 62 in EP 22216126.7 CACO2 Perm. 4.9 x 10⁻⁶ cm/sec Efflux ratio (BA/AB) 19.0 |
| 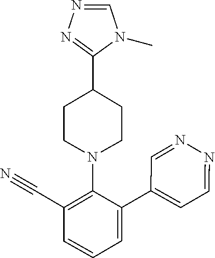 [SMILES: Cn1cnnc1C1CCN(c2c(C#N)cccc2-c2ccnnc2)CC1] Example 11 in WO 2024/020517 CACO2 Perm. 0.3 x 10⁻⁶ cm/sec Efflux ratio (BA/AB) 28.1 | 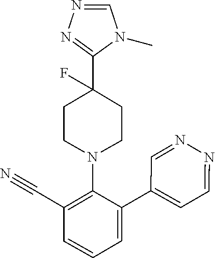 [SMILES: Cn1cnnc1C1(F)CCN(c2c(C#N)cccc2-c2ccnnc2)CC1] Example 52 in EP 22216126.7 CACO2 Perm. 15.0 x 10⁻⁶ cm/sec Efflux ratio (BA/AB) 1.1 |

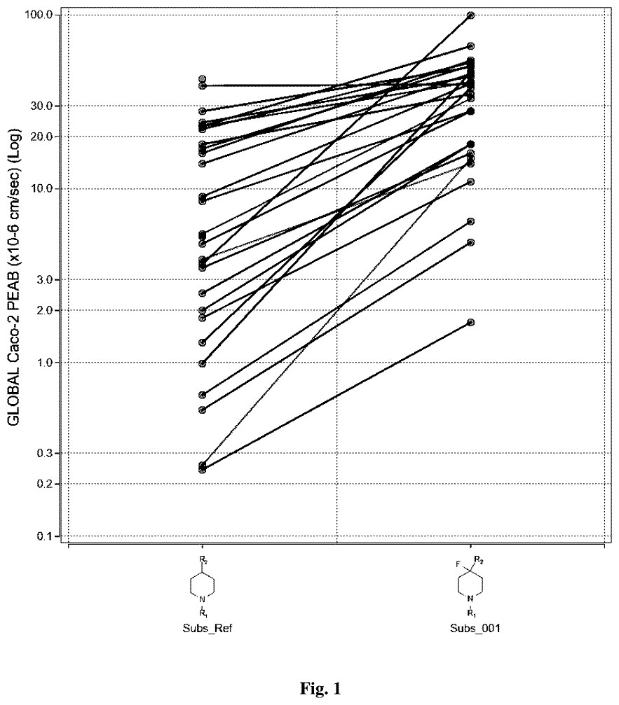

The increase in permeability in CACO2-Cells between the compared pairs of compounds is depicted in FIG. 1.

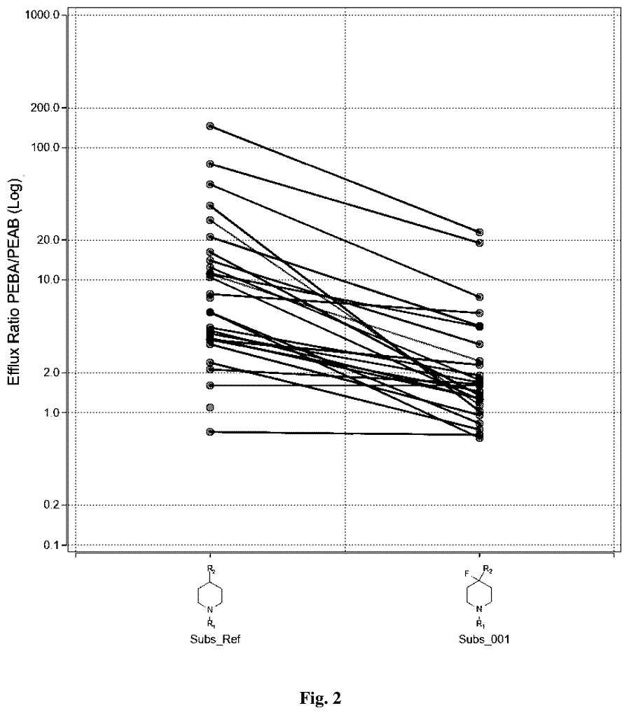

The decrease in efflux between the compared pairs of compounds is depicted in FIG. 2.

Consequently, compounds of the present invention are more viable for human use.

Compounds of the present invention differ structurally from Compound 14 in WO 2023/205173 in that the triazolyl ring in the 4-position of the piperidyl ring does not contain an amino substituent. Furthermore, the 4-position of the piperidyl ring is further substituted with fluoro. Still furthermore, the phenyl ring attached to the 1-position of the piperidyl has four substituents instead of three.

Compounds of the present invention differ structurally from the compounds in WO 2024/020517 including QP5020/Compound (1) in that the 4-position of the piperidyl ring is substituted with fluoro in addition to the triazolyl ring. Furthermore, the phenyl ring attached to the 1-position of the piperidyl ring has four substituents, with a heteroaryl substituent at the para-position relative to the piperidyl ring. This differs to compounds disclosed in WO 2024/020517 which have three substituents or a fourth heteroaryl substituent at the metaposition relative to the piperidyl ring, such as Compound 1, Compound 35 and Compound 44.

These structural differences between compounds of the present invention and the prior art unexpectedly lead to a favourable combination of (i) potent inhibition of QPCT and QPCTL, (ii) potent inhibition of QPCT/L in cells relevant for, but not limited to, lung diseases or cancer, (iii) appropriate membrane permeability and a low in vitro efflux, (iv) no or still acceptable induction of CYP3A4 mRNA levels and (v) improved stability in murine hepatocytes which facilitates preclinical compound evaluation and selection.

Compounds of the invention are thus superior to those disclosed in the prior art in terms of the combination of the following parameters:
potent inhibition of QPCT and QPCTL (Assay A)
potent inhibition of QPCT/L in cells relevant for, but not limited to, lung diseases or cancer (Assay B)
appropriate membrane permeability and a low in vitro efflux (Assay C)
no or still acceptable induction of CYP3A4 mRNA levels (Assay D)
improved stability in murine hepatocytes which facilitates preclinical compound evaluation and selection (Assay E)

The present invention provides novel compounds according to formula (I)

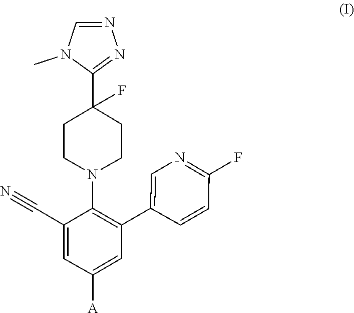

wherein
A is HOC(CH₃)₂C,

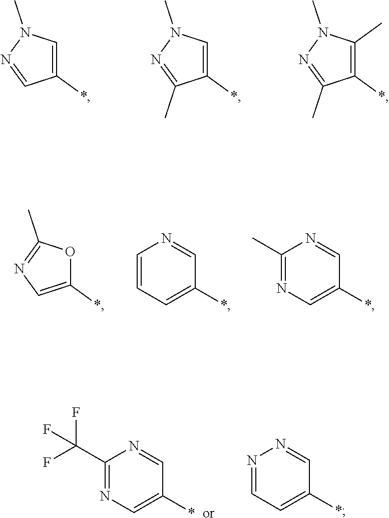

or a salt thereof, particularly a pharmaceutically acceptable salt thereof.

Particularly preferred is the compound according to formula (I) selected from the group consisting of

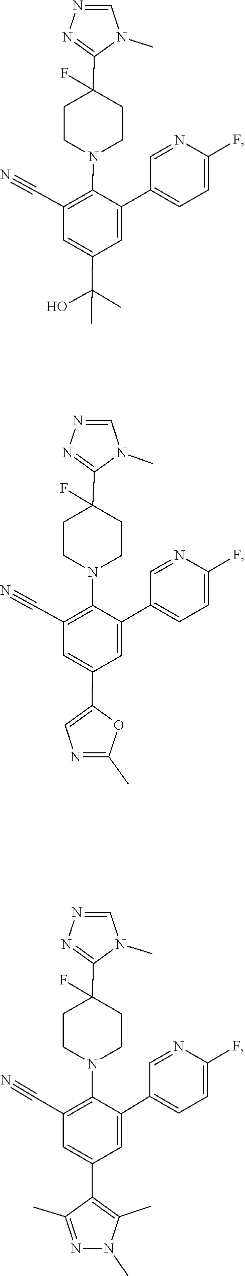 [SMILES: Cn1cnnc1C1(F)CCN(c2c(C#N)cc(C(C)(C)O)cc2-c2ccc(F)nc2)CC1 ; Cc1ncc(-c2cc(C#N)c(N3CCC(F)(c4nncn4C)CC3)c(-c3ccc(F)nc3)c2)o1 ; Cc1nn(C)c(C)c1-c1cc(C#N)c(N2CCC(F)(c3nncn3C)CC2)c(-c2ccc(F)nc2)c1]

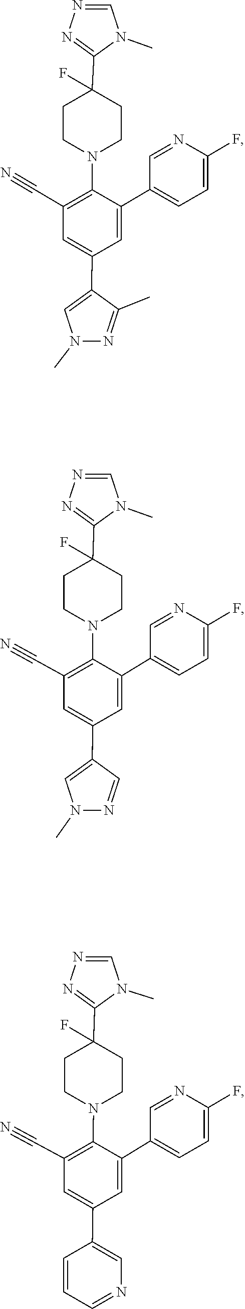 [SMILES: Cc1nn(C)cc1-c1cc(C#N)c(N2CCC(F)(c3nncn3C)CC2)c(-c2ccc(F)nc2)c1 ; Cn1cc(-c2cc(C#N)c(N3CCC(F)(c4nncn4C)CC3)c(-c3ccc(F)nc3)c2)cn1 ; Cn1cnnc1C1(F)CCN(c2c(C#N)cc(-c3cccnc3)cc2-c2ccc(F)nc2)CC1]

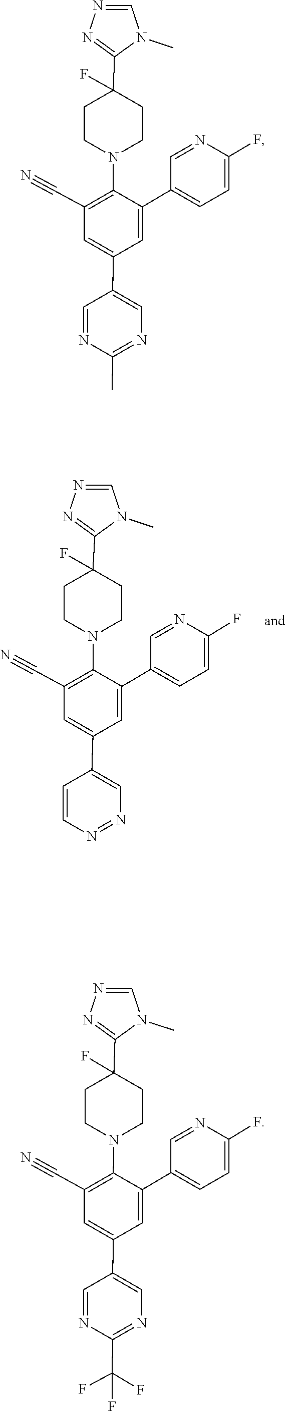 [SMILES: Cc1ncc(-c2cc(C#N)c(N3CCC(F)(c4nncn4C)CC3)c(-c3ccc(F)nc3)c2)cn1 ; Cn1cnnc1C1(F)CCN(c2c(C#N)cc(-c3ccnnc3)cc2-c2ccc(F)nc2)CC1 ; Cn1cnnc1C1(F)CCN(c2c(C#N)cc(-c3cnc(C(F)(F)F)nc3)cc2-c2ccc(F)nc2)CC1]

Particularly preferred is the compound according to formula (I) selected from the group consisting of example 1, example 2, example 3, example 4, example 5 and example 6, example 7, example 8 and example 9 as described hereinafter in EXAMPLES.

Particularly preferred is the compound according to formula (I) selected from the group consisting of example 1 and example 4, as described hereinafter in EXAMPLES.

Particularly preferred is the compound according to formula (I) selected from the group consisting of example 1, example 2, example 4, example 5 and example 6, as described hereinafter in EXAMPLES.

The present invention provides novel piperidinylpyridinylcarbonitrile derivatives of formula (I) that are surprisingly potent QPCT/L inhibitors.

Another aspect of the invention refers to compounds according to formula (I) as surprisingly having potent inhibition of QPCT/L in cells relevant for, but not limited to, lung diseases or cancer.

Another aspect of the invention refers to compounds according to formula (I) as surprisingly cellular potent QPCT/L inhibitors having appropriate membrane permeability, low in vitro efflux and low DDI perpetrator risk due to an appropriate CYP induction profile.

Another aspect of the invention refers to pharmaceutical compositions, containing at least one compound according to formula (I) optionally together with one or more inert carriers and/or diluents.

A further aspect of the present invention refers to compounds according to formula (I), for the use in the prevention and/or treatment of disorders associated with QPCT/L inhibition.

Another aspect of the invention refers to processes of manufacture of the compounds of the present invention.

Further aspects of the present invention will become apparent to the skilled artisan directly from the foregoing and following description and the examples.

### USED TERMS AND DEFINITIONS

### General Definitions

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁-6-alkyl means an alkyl group or radical having 1 to 6 carbon atoms. In general in groups like HO, H₂N, (O)S, (O)₂S, NC (cyano), HOOC, F₃C or the like, the skilled artisan can see the radical attachment point(s) to the molecule from the free valences of the group itself. For combined groups comprising two or more subgroups, the last named subgroup is the radical attachment point, for example, the substituent “aryl-C₁₋₃-alkylene” means an aryl group which is bound to a C₁₋₃-alkyl-group, the latter of which is bound to the core or to the group to which the substituent is attached.

In case a compound of the present invention is depicted in the form of a chemical name and as a formula, in case of any discrepancy the formula shall prevail. An asterisk may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

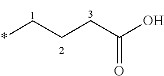 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

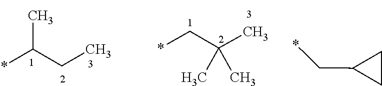

The asterisk may be used in sub-formulas to indicate the bond which is connected to the core molecule as defined.

The term “substituted” as used herein, means that one or more hydrogens on the designated atom are replaced by a group selected from a defined group of substituents, provided that the designated atom's normal valence is not exceeded, and that the substitution results in a stable compound. Likewise, the term “substituted” may be used in connection with a chemical moiety instead of a single atom, e.g. “substituted alkyl”, “substituted aryl” or the like.

Unless specifically indicated, throughout the specification and the appended claims, a given chemical formula or name shall encompass tautomers and all stereo, optical and geometrical isomers (e.g. enantiomers, diastereomers, E/Z isomers etc . . . ) and racemates thereof as well as mixtures in different proportions of the separate enantiomers, mixtures of diastereomers, or mixtures of any of the foregoing forms where such isomers and enantiomers exist, as well as solvates thereof such as for instance hydrates.

Unless specifically indicated, also “pharmaceutically acceptable salts” as defined in more detail below shall encompass solvates thereof such as for instance hydrates.

In general, substantially pure stereoisomers can be obtained according to synthetic principles known to a person skilled in the field, e.g. by separation of corresponding mixtures, by using stereochemically pure starting materials and/or by stereoselective synthesis. It is known in the art how to prepare optically active forms, such as by resolution of racemic forms or by synthesis, e.g. starting from optically active starting materials and/or by using chiral reagents.

Enantiomerically pure compounds of this invention or intermediates may be prepared via asymmetric synthesis, for example by preparation and subsequent separation of appropriate diastereomeric compounds or intermediates which can be separated by known methods (e.g. by chromatographic separation or crystallization) and/or by using chiral reagents, such as chiral starting materials, chiral catalysts or chiral auxiliaries.

Further, it is known to the person skilled in the art how to prepare enantiomerically pure compounds from the corresponding racemic mixtures, such as by chromatographic separation of the corresponding racemic mixtures on chiral stationary phases; or by resolution of a racemic mixture using an appropriate resolving agent, e.g. by means of diastereomeric salt formation of the racemic compound with optically active acids or bases, subsequent resolution of the salts and release of the desired compound from the salt; or by derivatization of the corresponding racemic compounds with optically active chiral auxiliary reagents, subsequent diastereomer separation and removal of the chiral auxiliary group; or by kinetic resolution of a racemate (e.g. by enzymatic resolution); by enantioselective crystallization from a conglomerate of enantiomorphous crystals under suitable conditions; or by (fractional) crystallization from a suitable solvent in the presence of an optically active chiral auxiliary.

The phrase “pharmaceutically acceptable” is employed herein to refer to those compounds, materials, compositions, and/or dosage forms which are, within the scope of sound medical judgment, suitable for use in contact with the tissues of human beings without excessive toxicity, irritation, allergic response, or other problem or complication, and commensurate with a reasonable benefit/risk ratio.

As used herein, “pharmaceutically acceptable salt” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like.

For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent such as ether, ethyl acetate, ethanol, isopropanol, or acetonitrile, or a mixture thereof.

Salts of other acids than those mentioned above which for example are useful for purifying or isolating the compounds of the present invention (e.g. trifluoro acetate salts,) also comprise a part of the invention.

The term halogen denotes fluorine, chlorine, bromine and iodine.

The term “C₁₋ₙ-alkyl”, wherein n is an integer selected from 2, 3, 4, 5 or 6, preferably 4, 5, or 6, either alone or in combination with another radical, denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₖ-cycloalkyl”, wherein k is an integer selected from 3, 4, 5, 7 or 8, preferably 4, 5 or 6, either alone or in combination with another radical, denotes a cyclic, saturated, unbranched hydrocarbon radical with 3 to k C atoms. For example the term C₃₋₇-cycloalkyl includes cyclopropyl, cyclobutyl, cyclopentyl, cyclohexyl and cycloheptyl.

The term “halo” added to an “alkyl”, “alkylene” or “cycloalkyl” group (saturated or unsaturated) defines an alkyl, alkylene or cycloalkyl group wherein one or more hydrogen atoms are replaced by a halogen atom selected from among fluorine, chlorine or bromine, preferably fluorine and chlorine, particularly preferred is fluorine. Examples include: H₂FC—, HF₂C—, F₃C—.

The term “mono-heteroaryl ring” means a monocyclic aromatic ring system, containing one or more heteroatoms selected from N, O or S, consisting of 5 to 6 ring atoms.

The term “mono-heteroaryl ring” is intended to include all the possible isomeric forms. Thus, the term “mono-heteroaryl ring” includes the following exemplary structures (not depicted as radicals as each form is optionally attached through a covalent bond to any atom so long as appropriate valences are maintained):

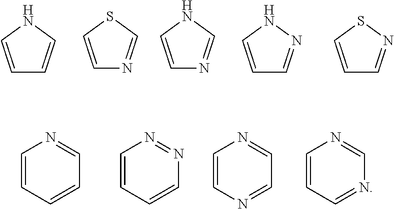

The term “fused bicyclic-heteroaryl ring” means a bicyclic aromatic ring system, containing one or more heteroatoms selected from N, O or S, consisting of 9 to 10 ring atoms. The term “fused bicyclic-heteroaryl ring” is intended to include all the possible isomeric forms. Thus, the term “bicyclic-heteroaryl ring” includes the following exemplary structures (not depicted as radicals as each form is optionally attached through a covalent bond to any atom so long as appropriate valences are maintained):

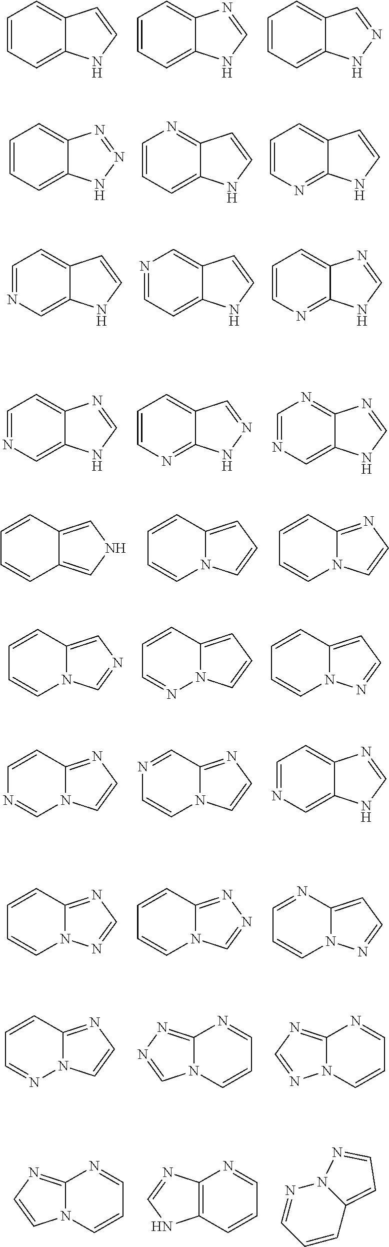

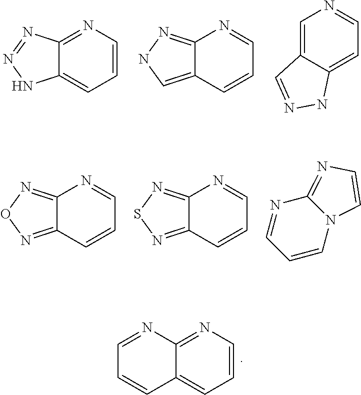

The term phenyl refers to the radical of the following ring:

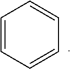

The term pyridyl refers to the radical of the following ring:

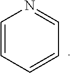

The term pyridazinyl refers to the radical of the following ring:

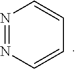

The term pyrimidyl refers to the radical of the following ring:

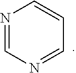

The term pyrazolyl refers to the radical of the following ring:

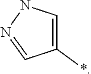

The term oxazolyl refers to the radical of the following ring:

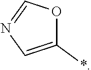

The term pyrazolopyridinyl refers to the radical of the following ring:

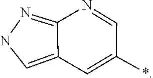

Many of the terms given above may be used repeatedly in the definition of a formula or group and in each case have one of the meanings given above, independently of one another.

### BIOLOGICAL ASSAYS

Evaluation of inhibitory activity on QPCT and QPCTL

### Assay A: Biochemical QPCT and QPCTL Activity Assay

The activity of the compounds of the invention may be demonstrated using the following biochemical enzyme activity assay:

QPCT or QPCTL dependent conversion of N-terminal glutamine to pyroglutamate of CD47 was monitored via MALDI-TOF MS. Test compounds were dissolved in 100% DMSO and serially diluted into clear 1,536-well microtiter plates. Enzymatic reactions were set up in assay buffer containing 20 mM Tris pH 7.5, 0.1 mM TCEP, 0.01% BSA, and 0.001% Tween20. 2.5 μL of 2× concentrated QPCTL (in-house) or QPCT (Origine #TP700028) enzyme in assay buffer (0.5 nM final concentration, columns 1-23) or plain assay buffer (columns 24) were added to each well. The plates were incubated for 10 min in a humidified incubator at 24° C. Subsequently, 2.5 μL of CD47 peptide substrate surrogate (¹⁹QLLFNKTKSVEFTFC³³) was added to each well (final concentration: 10 μM for QPCTL/20 μM for QPCT). The plates were mixed for 30 sec at 1,000 rpm and subsequently incubated for 40 min in a humidified incubator at 24° C. After incubation, the enzymatic reaction was stopped by adding 1 μL containing stable isotope labeled internal standard peptide ¹⁹[Pyr]LLFN(K)TKSVEFTFC³³ (final concentration 4.0 μM) as well as SEN177 (final concentration 10 μM). The plates were sealed with an adhesive foil, mixed for 30 s at 1,000 rpm and stored at room temperature until preparation of the MALDI target plates. MALDI target plates were prepared as described previously.1 Mass spectra were acquired with a rapifleX MALDI-TOF/TOF instrument tracking the signals of the product (¹⁹[Pyr]LLFNKTKSVEFTFC³³, m/z 1,787.9037) as well as internal standard (¹⁹[Pyr]LLFN(K)TKSVEFTFC³³, m/z 1,795.9179) peptide. QPCT or QPCTL activity was monitored by calculating the ratio between product and internal standard signals followed by normalization to high (100% activity) and low (0% activity) controls. Determination of compound potencies was obtained by fitting the dose-response data to a four-parameter logistical equation.

TABLE 2
Biological data for compounds of the
invention as obtained in Assay A.

| | Inhibition of | Inhibition of |
|---|---|---|
| | QPCTL: | QPCT: |
| Example | IC₅₀ [nM] | IC₅₀ [nM] |
| 1 | 1 | 1 |
| 2 | 2 | 4 |
| 3 | 4 | 5 |
| 4 | 2 | 3 |
| 5 | 2 | 2 |
| 6 | 3 | 4 |
| 7 | 3 | 4 |
| 8 | 2 | 3 |
| 9 | 3 | 6 |

TABLE 3
Biological data for prior art compounds as obtained in Assay A.

| | | Inhibition of | Inhibition of |
|---|---|---|---|
| Prior art | | QPCTL: | QPCT: |
| Compound | Reference | IC₅₀ [nM] | IC₅₀ [nM] |
| Compound 14 | WO 2023/205173 | 1 | <1 |
| QP5038 | STTT 2023 | 2 | 5 |
| Compound | WO 2024/020517/ | 2 | 7 |
| (1)/QP5020 | STTT2023 | | |
| Compound 35 | WO 2024/020517 | 2 | 4 |
| Compound 44 | WO 2024/020517 | 1 | 5 |

### Assay B: SIRPα Signalling Assay (Using Either Raji or A549 Cells)

The activity of the compounds of the invention may be demonstrated using the following SIRPα signalling assay that measures SIRPα engagement induced by CD47 presented via cell-cell interaction. Two cell types are independently used: the Raji cell line (lymphoblast-like human cell line derived from B lymphocytes from a Burkitt's lymphoma patient in 1963) and A549 cells (adenocarcinomic human alveolar basal epithelial cells).

Test compounds were dissolved in 100% DMSO and serially diluted into a white 384-well microtiter cell culture plate (PerkinElmer #60076780 in case of Raji assay; PDL-coated plates Greiner #781945 in case of A549 assay). 5000 Raji cells (ATCC #CC86) or 5000 A549 cells (ATCC #CCL-185) in Assay Complete Cell Plating reagent 30 (DiscoverX 93-0563R30B) were added per well. The assay plate was incubated for 48 h at 37° C., 95% humidity and 5% CO₂. 15000 reporter cells (Jurkat PathHunter SIRPαV1, DiscoverX #93-1135C19) were added to each well, and the plate was incubated for 5 h at 37° C., 95% humidity and 5% CO₂. Bioassay reagent 1 of the PathHunter Bioassay detection kit (DiscoverX 93-0001) was added to each well of the plate using a multichannel pipette followed by a 15 min incubation at room temperature. Afterwards bioassay reagent 2 was added followed by 60 min incubation at room temperature (incubation in the dark).

The analysis of the data was performed using the luminescence signal generated by betagalactosidase in the PathHunter reporter cell line. The luminescence measurement was done using a Pherastar Multi-Mode Reader. Dose-response curves & IC₅₀ data were calculated with 4-parameter sigmoidal dose response formula.

TABLE 4
Biological data for compounds of the
invention as obtained in Assay B.

| | Inhibition of | Inhibition of |
|---|---|---|
| | SIRPα signalling | SIRPα signalling |
| | induced by Raji | induced by A549 |
| | cells: | cells: |
| Example | IC₅₀ [nM] | IC₅₀ [nM] |
| 1 | 26 | 88 |
| 2 | 180 | 36 |
| 3 | 330 | 45 |
| 4 | 90 | 26 |
| 5 | 52 | 25 |
| 6 | 139 | 33 |
| 7 | 100 | 37 |
| 8 | 267 | 651 |
| 9 | 108 | 67 |

TABLE 5
Biological data for prior art compounds as obtained in Assay B.

| | | Inhibition of | Inhibition of |
|---|---|---|---|
| | | SIRPα | SIRPα |
| | | signalling | signalling |
| | | induced by Raji | induced by A549 |
| Prior art | | cells: | cells: |
| Compound | Reference | IC₅₀ [nM] | IC₅₀ [nM] |
| Compound 14 | WO 2023/205173 | 4 | 4 |
| QP5038 | STTT 2023 | 21 | 6 |
| Compound | WO 2024/020517/ | 51 | 8 |
| (1)/QP5020 | STTT2023 | | |
| Compound 35 | WO 2024/020517 | 28 | 9 |
| Compound 44 | WO 2024/020517 | 121 | 67 |

### Evaluation of Permeability

### Assay C: Permeability in CACO-2 Cells

Caco-2 cells (1-2×10⁵ cells/1 cm² area) are seeded on filter inserts (Costar transwell polycarbonate or PET filters, 0.4 μm pore size) and cultured (DMEM) for 10 to 25 days. Compounds are dissolved in appropriate solvent (like DMSO, 1-20 mM stock solutions). Stock solutions are diluted with HTP-4 buffer (128.13 mM NaCl, 5.36 mM KCl, 1 mM MgSO₄, 1.8 mM CaCl₂), 4.17 mM NaHCO₃, 1.19 mM Na₂HPO_{4×}7H₂O, 0.41 mM NaH₂PO₄xH₂O, 15 mM HEPES, 20 mM glucose, 0.25% BSA, pH 7.2) to prepare the transport solutions (0.1-300 μM compound, final DMSO<=0.5%). The transport solution (TL) is applied to the apical or basolateral donor side for measuring A-B or B-A permeability (3 filter replicates), respectively. Samples are collected at the start and end of experiment from the donor and at various time intervals for up to 2 hours also from the receiver side for concentration measurement by HPLC-MS/MS or scintillation counting. Sampled receiver volumes are replaced with fresh receiver solution.

Efflux ratio (ER)=permeability B-A/permeability A-B

TABLE 7
Biological data for compounds of the
invention as obtained in Assay C.

| | Permeability A-B | Efflux |
|---|---|---|
| Example | [10⁻⁶ cm/s] | Ratio |
| 1 | 4.2 | 11.9 |
| 2 | 26.0 | 3.3 |
| 3 | 11.0 | 7.8 |
| 4 | 5.0 | 8.8 |
| 5 | 5.4 | 6.7 |
| 6 | 19.0 | 4.6 |
| 7 | 4.8 | 7.9 |
| 8 | 0.7 | 20.3 |
| 9 | 4.8 | 5.2 |

TABLE 8
Biological data for prior art compounds as obtained in Assay C.

| Prior art | | Permeability A-B | Efflux |
|---|---|---|---|
| Compound | Reference | [10⁻⁶ cm/s] | Ratio |
| Compound 14 | WO 2023/205173 | 0.7 | 22.2 |
| QP5038 | STTT 2023 | 61.0 | 0.8 |
| Compound | WO 2024/020517/ | 28.0 | 2.4 |
| (1)/QP5020 | STTT2023 | | |
| Compound 35 | WO 2024/020517 | 4.9 | 12.2 |
| Compound 44 | WO 2024/020517 | 0.5 | 47.9 |

### Evaluation of CYP3A4 Induction

### Assay D: CYP Induction Screening Assay in Primary Human Hepatocytes

Cryopreserved plateable human hepatocytes (single donor, BioIVT) were thawed and plated in Collagen-I coated 96-well-plates at a cell density of 0.07 million cells per well.

After a 6 h attachment period, the seeding medium was replaced by serum-free William's medium E supplemented with Matrigel (0.25 mg/ml) and allowed to recover overnight. 24 h post-seeding, serum-free Williams E medium containing the test compound at a final concentration of 10 μM and a final DMSO content of 0.1% and 0.1% DMSO (solvents treated control), respectively, was added to predefined wells. Exposure solutions were renewed after 24 h.

After 48 h of treatment in total, the effect of the test compounds on CYP3A4 mRNA expression was assessed using the QuantiGene Plex Gene Expression Assay. Hepatocytes were lysed and total RNA was extracted using the QuantiGene Sample Processing Kit according to the instructions of the manufacturer.

mRNA quantification was conducted using a customized QuantiGene Plex Panel to analyse CYP3A4 and the housekeeper genes RPL32, EIF4E2 and GUSB according to the instructions of the manufacturer and measured on a Luminex™ instrument. Signal was reported as median fluorescence intensity (MFI), which is proportional to the number of target RNA molecules present in the sample.

For calculation of CYP3A4 mRNA induction, the signal for CYP3A4 was normalized against the geometric mean of the signal obtained for the housekeeper genes for hepatocytes exposed to test compounds in relation to solvent-treated samples according to the following equation:

n-fold induction=(MFI CYP3A4 (treated)/X̅geo MFI (RPL32,EIF4E2,GUSB))/(MFI CYP3A4 (solvent control)/X̅geo MFI (RPL32,EIF4E2,GUSB))

TABLE 9
Biological data for compounds of the
invention as obtained in Assay D.

| | n-fold induction of |
|---|---|
| Example | CYP3A4 at 10 μM |
| 1 | 2.2 |
| 2 | 1.6 |
| 3 | 1.7 |
| 4 | 2.0 |
| 5 | 7.7 |
| 6 | 1.8 |
| 7 | 0.7 |
| 8 | 0.9 |
| 9 | 1.4 |

TABLE 10
Biological data for prior art compounds as obtained in Assay D.

| Prior art | | n-fold induction of |
|---|---|---|
| Compound | Reference | CYP3A4 at 10 μM |
| Compound 14 | WO 2023/205173 | 1.9 |
| QP5038 | STTT 2023 | 58.5 |
| Compound | WO 2024/020517/ | 3.4 |
| (1)/QP5020 | STTT 2023 | |
| Compound 35 | WO 2024/020517 | 3.0 |
| Compound 44 | WO 2024/020517 | 1.0 |

### Evaluation of Hepatic Stability (Mouse)

### Assay E: Stability in Murine Hepatocytes

The metabolic degradation of the test compound is assayed in a murine hepatocyte suspension.

Murine hepatocytes (typically cryopreserved) are incubated in an appropriate buffer system (e.g. Dulbecco's modified eagle medium plus 3.5 μg glucagon/500 mL, 2.5 mg insulin/500 mL and 3.75 mg/500 mL hydrocortison) containing 5% species serum.

Following a (typically) 30 min preincubation in an incubator (37° C., 10% CO2) 5 μl of test compound solution (80 μM; from 2 mM in DMSO stock solution diluted 1:25 with medium) are added into 395 μl hepatocyte suspension (cell density in the range 0.25-5 Mio cells/mL, typically 1 Mio cells/mL; final concentration of test compound 1 μM, final DMSO concentration 0.05%).

The cells are incubated for six hours (incubator, orbital shaker) and samples (2 μl) are taken at 0, 0.5, 1, 2, 4 and 6 hours. Samples are transferred into acetonitrile and pelleted by centrifugation (5 min). The supernatant is transferred to a new 96-deepwell plate, evaporated under nitrogen and resuspended.

### Decline of Parent Compound is Analyzed by HPLC-MS/MS

CLint is calculated as follows CL_INTRINSIC=Dose/AUC=(C0/CD)/(AUD+clast/k)×1000/60. C0: initial concentration in the incubation [μM], CD: cell density of vital cells [10e6cells/mL], AUD: area under the data [μM×h], clast: concentration of last data point [μM], k: slope of the regression line for parent decline [h−1].

The calculated in vitro hepatic intrinsic clearance can be scaled up to the intrinsic in vivo hepatic Clearance and used to predict hepatic in vivo blood clearance (CL) by the use of a liver model (well stirred model).

CL_INTRINSIC_INVIVO[ml/min/kg]=(CL_INTRINSIC[μL/min/10e6cells]×hepatocellularity[10e6cells/g liver]×liver factor[g/kg bodyweight])/1000

CL[ml/min/kg]=CL_INTRINSIC_INVIVO[ml/min/kg]×hepatic blood flow[ml/min/kg]/(CL_INTRINSIC_INVIVO[ml/min/kg]+hepatic blood flow[ml/min/kg])

Q_{H}[1%]=CL[ml/min/kg]/hepatic blood flow[ml/min/kg])

Hepatocellularity,mouse:120×10e6cells/g liver

Liver factor,mouse:55 g/kg bodyweight

Blood flow,mouse:90 ml/(min×kg)

TABLE 12
Biological data for compounds of the
invention as obtained in Assay E.

| | Mouse |
|---|---|
| | Hepatocyte |
| | Stability |
| Example | QH [%] |
| 1 | 23 |
| 2 | 15 |
| 3 | 25 |
| 4 | 21 |
| 5 | <12 |
| 6 | 35 |
| 7 | 48 |
| 8 | 20 |
| 9 | 20 |

TABLE 13
Biological data for prior art compounds as obtained in Assay E.

| | | Mouse |
|---|---|---|
| | | Hepatocyte |
| Prior art | | Stability |
| Compound | Reference | Q_{H} [%] |
| Compound 14 | WO 2023/205173 | 31 |
| QP5038 | STTT 2023 | 97 |
| Compound | WO 2024/020517/ | 82 |
| (1)/QP5020 | STTT 2023 | |
| Compound 35 | WO 2024/020517 | 16 |
| Compound 44 | WO 2024/020517 | 65 |

### Evaluation of Microsomal Clearance

### Microsomal Clearance:

The metabolic degradation of the test compound was assayed at 37° C. with pooled liver microsomes from various species. The final incubation volume of 60 μl per time point contains TRIS buffer pH 7.6 at room temperature (0.1 M), magnesium chloride (5 mM), microsomal protein (1 mg/mL for human and dog, 0.5 mg/mL for other species) and the test compound at a final concentration of 1 μM. Following a short preincubation period at 37° C., the reactions were initiated by addition of betanicotinamide adenine dinucleotide phosphate, reduced form (NADPH, 1 mM), and terminated by transferring an aliquot into solvent after different time points. After centrifugation (10000 g, 5 min), an aliquot of the supernatant was assayed by LC-MS/MS for the amount of parent compound. The half-life was determined by the slope of the semi-logarithmic plot of the concentration-time profile.

The intrinsic clearance (CL_INTRINSIC) is calculated by considering the amount of protein in the incubation:

$$CL\_INTRINSIC [ μl / min / mg protein ] = ( Ln 2 / ( half - life [ min ] * protein content [ mg / ml ] ) ) * 1000$$

$$CL\_INTRINSIC \_INVIVO [ ml / min / kg ] = ( CL\_INTRINSIC [ μL / min / mg protein ] \times MPPGL [ mg protein / g liver ] \times liver factor [ g / kg body weight ] ) / 1000$$

$$Qh [ \% ] = CL [ ml / min / kg ] / hepatic blood flow [ ml / min / kg ] )$$

Hepatocellularity, human: 120×10e6 cells/g liver
Liver factor, human: 25.7 g/kg bodyweight
Blood flow, human: 21 ml/(min×kg)

### Evaluation of Hepatocyte Clearance

### Human Hepatocyte Clearance

The metabolic degradation of a test compound is assayed in a human hepatocyte suspension. After recovery from cryopreservation, human hepatocytes are diluted in Dulbecco's modified eagle medium (supplemented with 3.5 μg glucagon/500 mL, 2.5 mg insulin/500 mL, 3.75 mg hydrocortisone/500 mL, 5% human serum) to obtain a final cell density of 1.0×10⁶ cells/mL.

Following a 30 minutes preincubation in a cell culture incubator (37° C., 10% CO₂), test compound solution is spiked into the hepatocyte suspension, resulting in a final test compound concentration of 1 μM and a final DMSO concentration of 0.05%.

The cell suspension is incubated at 37° C. (cell culture incubator, horizontal shaker) and samples are removed from the incubation after 0, 0.5, 1, 2, 4 and 6 hours. Samples are quenched with acetonitrile (containing internal standard) and pelleted by centrifugation. The supernatant is transferred to a 96-deepwell plate, and prepared for analysis of decline of parent compound by HPLC-MS/MS.

The percentage of remaining test compound is calculated using the peak area ratio (test compound/internal standard) of each incubation time point relative to the time point 0 peak area ratio. The log-transformed data are plotted versus incubation time, and the absolute value of the slope obtained by linear regression analysis is used to estimate in vitro half-life (T1/2).

In vitro intrinsic clearance (CLint) is calculated from in vitro T1/2 and scaled to whole liver using a hepatocellularity of 120×106 cells/g liver, a human liver per body weight of 25.7 g liver/kg as well as in vitro incubation parameters, applying the following equation:

CL_INTRINSIC_IN VIVO [mL/min/kg]=(CL_INTRINSIC [μL/min/106 cells]×hepatocellularity [106 cells/g liver]×liver factor [g/kg body weight])/1000

Hepatic in vivo blood clearance (CL) is predicted according to the well-stirred liver model considering an average liver blood flow (QH) of 20.7 mL/min/kg:

$$CL [ mL / min / kg ] = CL\_INTRINSIC \_IN VIVO [ mL / min / kg ] \times hepatic blood flow [ mL / min / kg ] / ( CL\_INTRINSIC \_IN VIVO [ mL / min / kg ] + hepatic blood flow [ mL / min / kg ] )$$

Results are expressed as percentage of hepatic blood flow:

$$QH [ \% ] = CL [ mL / min / kg ] / hepatic blood flow [ mL / min / kg ] )$$

### Evaluation of Plasma Protein Binding

Equilibrium dialysis technique is used to determine the approximate in vitro fractional binding of test compounds to plasma proteins applying Dianorm Teflon dialysis cells (micro 0.2). Each dialysis cell consists of a donor and an acceptor chamber, separated by an ultrathin semipermeable membrane with a 5 kDa molecular weight cutoff. Stock solutions for each test compound are prepared in DMSO at 1 mM and serially diluted to obtain a final test concentration of 1 μM. The subsequent dialysis solutions are prepared in plasma (supplemented with NaEDTA as anticoagulant), and aliquots of 200 μl test compound dialysis solution in plasma are dispensed into the donor (plasma) chambers. Aliquots of 200 μl dialysis buffer (100 mM potassium phosphate, pH 7.4, supplemented with up to 4.7% Dextran) are dispensed into the buffer (acceptor) chamber. Incubation is carried out for 2 hours under rotation at 37° C. for establishing equilibrium.

At the end of the dialysis period, aliquots obtained from donor and acceptor chambers, respectively, are transferred into reaction tubes and processed for HPLC-MS/MS analysis. Analyte concentrations are quantified in aliquots of samples by HPLC-MS/MS against calibration curves.

Percent bound is calculated using the formula:

$$\% bound = ( plasma concentration - buffer concentration / plasma concentration ) \times 100$$

### Evaluation of Solubility

Saturated solutions are prepared in well plates (format depends on robot) by adding an appropriate volume of selected aqueous media (typically in the range of 0.25-1.5 ml) into each well which contains a known quantity of solid drug substance (typically in the range 0.5-5.0 mg). The wells are shaken or stirred for a predefined time period (typically in a range of 2-24 h) and then filtered using appropriate filter membranes (typically PTFE-filters with 0.45 μm pore size). Filter absorption is avoided by discarding the first few drops of filtrate. The amount of dissolved drug substance is determined by UV spectroscopy. In addition, the pH of the aqueous saturated solution is measured using a glass-electrode pH meter.

### Evaluation of Metabolism in Human Hepatocytes In Vitro

The metabolic pathway of a test compound is investigated using primary human hepatocytes in suspension. After recovery from cryopreservation, human hepatocytes are incubated in Dulbecco's modified eagle medium containing 5% human serum and supplemented with 3.5 μg glucagon/500 ml, 2.5 mg insulin/500 ml and 3.75 mg/500 ml hydrocortisone.

Following a 30 min preincubation in a cell culture incubator (37° C., 10% CO₂), test compound solution is spiked into the hepatocyte suspension to obtain a final cell density of 1.0*10⁶ to 4.0*10⁶ cells/ml (depending on the metabolic turnover rate of the compound observed with primary human hepatocytes), a final test compound concentration of 10 PM, and a final DMSO concentration of 0.05%.

The cells are incubated for six hours in a cell culture incubator on a horizontal shaker, and samples are removed from the incubation after 0, 0.5, 1, 2, 4 or 6 hours, depending on the metabolic turnover rate. Samples are quenched with acetonitrile and pelleted by centrifugation. The supernatant is transferred to a 96-deepwell plate, evaporated under nitrogen and resuspended prior to bioanalysis by liquid chromatography-high resolution mass spectrometry for identification of putative metabolites.

The structures are assigned tentatively based on Fourier-Transform-MS” data. Metabolites are reported as percentage of the parent in human hepatocyte incubation with a threshold of ≥4%.

### Evaluation of Pharmacokinetic Characteristics

The test compound is administered either intravenously or orally to the respective test species. Blood samples are taken at several time points post application of the test compound, anticoagulated and centrifuged.

The concentration of analytes—the administered compound and/or metabolites—are quantified in the plasma samples. PK parameters are calculated using non compartment methods. AUC and Cmax are normalized to a dose of 1 μmol/kg.

### Method of Treatment

The present invention is directed to compounds of general formula (I) which are useful in the prevention and/or treatment of a disease and/or condition associated with or modulated by QPCT/L activity, including but not limited to the treatment and/or prevention of cancer, fibrotic diseases, neurodegenerative diseases, atherosclerosis, infectious diseases, chronic kidney diseases.

The compounds of general formula (I) are useful for the prevention and/or treatment of: (1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.

(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.

(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenois carcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, nonHodgkin'lymphoma (NHL), urothelial cancer, or peritoneal cancer.

(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.

(5) Neurodegenerative disorders such as amyotrophic lateral sclerosis, multiple sclerosis, Parkinson's disease, Alzheimer's disease, Huntington's disease, multiple system atrophy, or prion diseases.

Accordingly, the present invention relates to a compound of general formula (I) or a pharmaceutically acceptable salt thereof for use as a medicament.

Furthermore, the present invention relates to the use of a compound of general formula (I) for the treatment and/or prevention of a disease and/or condition associated with or modulated by QPCT/L activity.

Furthermore, the present invention relates to the use of a compound of general formula (I) or a pharmaceutically acceptable salt thereof or a pharmaceutical composition thereof for the treatment and/or prevention of cancer, fibrotic diseases, neurodegenerative diseases, atherosclerosis, infectious diseases, chronic kidney diseases.

Furthermore, the present invention relates to the use of a compound of general formula (I) or a pharmaceutically acceptable salt thereof or a pharmaceutical composition thereof for the treatment and/or prevention of: (1) Pulmonary fibrotic diseases such as pneumonitis or interstitial pneumonitis associated with collagenosis, e g. lupus erythematodes, systemic scleroderma, rheumatoid arthritis, polymyositis and dermatomysitis, idiopathic interstitial pneumonias, such as pulmonary lung fibrosis (IPF), non-specific interstitial pneumonia, respiratory bronchiolitis associated interstitial lung disease, desquamative interstitial pneumonia, cryptogenic orgainizing pneumonia, acute interstitial pneumonia and lymphocytic interstitial pneumonia, lymangioleiomyomatosis, pulmonary alveolar proteinosis, Langerhan's cell histiocytosis, pleural parenchymal fibroelastosis, interstitial lung diseases of known cause, such as interstitial pneumonitis as a result of occupational exposures such as asbestosis, silicosis, miners lung (coal dust), farmers lung (hay and mould), Pidgeon fanciers lung (birds) or other occupational airbourne triggers such as metal dust or mycobacteria, or as a result of treatment such as radiation, methotrexate, amiodarone, nitrofurantoin or chemotherapeutics, or for granulomatous disease, such as granulomatosis with polyangitis, Churg-Strauss syndrome, sarcoidosis, hypersensitivity pneumonitis, or interstitial pneumonitis caused by different origins, e g. aspiration, inhalation of toxic gases, vapors, bronchitis or pneumonitis or interstitial pneumonitis caused by heart failure, X-rays, radiation, chemotherapy, M. boeck or sarcoidosis, granulomatosis, cystic fibrosis or mucoviscidosis, or alpha-I-antitrypsin deficiency.

(2) Other fibrotic diseases such as hepatic bridging fibrosis, liver cirrhosis, non-alcoholic steatohepatitis (NASH), atrial fibrosis, endomyocardial fibrosis, old myocardial infarction, glial scar, arterial stiffness, arthrofibrosis, Dupuytren's contracture, keloid, scleroderma/systemic sclerosis, mediastinal fibrosis, myelotibrosis, Peyronie's disease, nephrogenic systemic fibrosis, retroperitoneal fibrosis, adhesive capsulitis; spontaneous acute exacerbations in pulmonary fibrosis and progressive pulmonary fibrosis or induced by infection, microaspiration, surgical lung biopsy, surgical resection, bronchoscopy (BAL, cryobiopsy), air pollution, prior exacerbation and medications.

(3) Leukemia, acute myeloid leukemia (AML), acute promyelocytic leukemia (APL), chronic myeloid leukemia (CML), acute lymphoblastic leukemia (ALL), T-cell acute lymphoblastic leukemia (T-ALL), lymphoma, B-cell lymphoma, T-cell lymphoma, Hodgkin's disease, non-Hodgkin's lymphoma (NHL), hairy cell lymphoma, Burkett's lymphoma, multiple myeloma (MM), myelodysplastic syndrome, solid cancer, lung cancer, adenocarcinoma, small cell lung cancer (SCLC), non-small cell lung cancer (NSCLC), mediastinum cancer, peritoneal cancer, mesothelioma, gastrointestinal cancer, gastric cancer, stomach cancer, bowel cancer, small bowel cancer, large bowel cancer, colon cancer, colon adenocarcinoma, colon adenoma, rectal cancer, colorectal cancer, leiomyosarcoma, breast cancer, gynaecological cancer, genito-urinary cancer, ovarian cancer, endometrial cancer, cervical cancer, prostate cancer, testicular cancer, seminoma, teratocarcinoma, liver cancer, kidney cancer, bladder cancer, urothelial cancer, biliary tract cancer, pancreatic cancer, exocrine pancreatic carcinoma, esophageal cancer, nasopharyngeal cancer, head and neck squamous cell carcinoma (HNSCC), skin cancer, squamous cancer, squamous cell carcinoma, Kaposi's sarcoma, melanoma, malignant melanoma, xeroderma pigmentosum, keratoacanthoma, bone cancer, bone sarcoma, osteosarcoma, rhabdomyosarcoma, fibrosarcoma, thyroid gland cancer, thyroid follicular cancer, adrenal gland cancer, nervous system cancer, brain cancer, astrocytoma, neuroblastoma, glioma, schwannoma, glioblastoma, or sarcoma, gastrointestinal cancer, gastric cancer, stomach cancer, esophageal cancer, head and neck squamous cell carcinoma (HNSCC), breast cancer, colorectal cancer, bowel cancer, large bowel cancer, colon cancer, colon adenocarcinoma, colon adenoma, rectal cancer, ovarian cancer, pancreatic cancer, exocrine pancreatic carcinoma, leukemia, acute myeloid leukemia (AML), myelodysplastic syndrome, lymphoma, B-cell lymphoma, nonHodgkin's lymphoma (NHL), urothelial cancer, or peritoneal cancer.

(4) Inflammatory, auto-immune or allergic diseases and conditions such as asthma, pediatric asthma, allergic bronchitis, alveolitis, hyperreactive airways, allergic conjunctivitis, bronchiectasis, adult respiratory distress syndrome, bronchial and pulmonary edema, bronchitis or pneumonitis, non-allergic asthma, chronic obstructive pulmonary disease (COPD), acute bronchitis, chronic bronchitis, pulmonary emphysema; autoimmune diseases, such as rheumatoid arthritis, Graves' disease, Sjogren's syndrome psoriatic arthritis, multiple sclerosis, systemic lupus Erythematosus, inflammatory bowel diseases, such as Crohn's disease and ulcerative colitis, scleroderma; psoriasis (including T-cell mediated psoriasis) and inflammatory dermatoses such as an dermatitis, eczema, atopic dermatitis, allergic contact dermatitis, urticaria; vasculitis (e g, necrotizing, cutaneous, and hypersensitivity vasculitis), or erythemanodosum.

(5) Neurodegenerative disorders such as amyotrophic lateral sclerosis, multiple sclerosis, Parkinson's disease, Alzheimer's disease, Huntington's disease, multiple system atrophy, or prion diseases.

In a further aspect the present invention relates to a compound of general formula (I) or a pharmaceutically acceptable salt thereof or a pharmaceutical composition thereof for use in the treatment and/or prevention of above-mentioned diseases and conditions.

In a further aspect the present invention relates to the use of a compound of general formula (I) or a pharmaceutically acceptable salt thereof or a pharmaceutical composition thereof for the preparation of a medicament for the treatment and/or prevention of abovementioned diseases and conditions.

In a further aspect of the present invention the present invention relates to methods for the treatment or prevention of above-mentioned diseases and conditions, which method comprises the administration of an effective amount of a compound of general formula (I) or a pharmaceutically acceptable salt thereof or a pharmaceutical composition thereof to a human being.

### Combination Therapy

The compounds of the invention may further be combined with one or more, preferably one additional therapeutic agent. According to one embodiment the additional therapeutic agent is selected from the group of therapeutic agents useful in the treatment of diseases or conditions described hereinbefore, in particular associated with cancer, fibrotic diseases, Alzheimer's diseases, atherosclerosis, infectious diseases, chronic kidney diseases and auto-immune disease.

Additional therapeutic agents that are suitable for such combinations include in particular those, which, for example, potentiate the therapeutic effect of one or more active substances with respect to one of the indications mentioned and/or allow the dosage of one or more active substances to be reduced.

Therefore, a compound of the invention may be combined with one or more additional therapeutic agents selected from the group consisting of chemotherapy, targeted cancer therapy, cancer immunotherapy, irradiation, antifibrotic agents, anti-tussive agents, antiinflammatory agents, anti-atopic dermatitis, and broncho dilators.

Chemotherapy is a type of cancer therapy that uses one or more chemical anti-cancer drugs, such as cytostatic or cytotoxic substances, cell proliferation inhibitors, anti-angiogenic substances and the like. Examples include folic acid (Leucovorin), 5-Fluorouracil, Irinotecan, Oxaliplatin, cis-platin Azacytidine, gemcitabine, alkylation agents, antimitotic agents, taxanes and further state-of-the-art or standard-of-care compounds.

Targeted therapy is a type of cancer treatment that uses drugs to target specific genes and proteins that help cancer cells survive and grow. Targeted therapy includes agents such as inhibitors of growth factors (e.g. platelet derived growth factor (PDGF), fibroblast growth factor (FGF), vascular endothelial growth factor (VEGF), epidermal growth factor (EGF), insulin-like growth factors (IGF), human epidermal growth factor (HER, e.g. HER2, HER3, HER4) and hepatocyte growth factor), tyrosine-kinases, KRAS, BRAF, BCR-ABL, mTOR, cyclin-dependent kinases, or MDM2.

Cancer immunotherapy is a type of therapy that uses substances to stimulate or suppress the immune system to help the body fight cancer. Cancer immunotherapy includes a therapeutic antibody, such as: anti-Her2 antibody, an anti-EGFR antibody, and an anti-PDGFR antibody; an anti-GD2 (Ganglioside G2) antibody. Examples include Dinutuximab, Olaratumab, Trastuzumab, Pertuzumab, Ertumaxomab, Cetuximab, Necitumumab, Nimotuzumab, Panitumumab, or rituximab. Cancer immunotherapy also includes a therapeutic antibody which is a checkpoint inhibitor, such as an anti PD1, anti PD-L1 antibody or CTLA4 inhibitor. Examples include Atezolizumab, Avelumab, and Durvalumab, Ipilimumab, nivolumab, or pembrolizumab. Cancer immunotherapy also includes agents which target (inhibit) the CD47-SIRPα signaling axis, such as agents which bind to CD47 or SIRPα. Non-limiting examples include antibodies such as anti-CD47 antibodies and anti-SIRPα antibodies, and recombinant Fc-fusion proteins such as CD47-Fc and SIRPα-Fc. Cancer immunotherapy also includes STING-targeting agent, or T cell engagers, such as blinatumomab.

Antifibrotic agents are for example nintedanib, pirfenidone, phosphodiesterase-IV (PDE4) inhibitors such as roflumilast or specific PDE4b inhibitors like BI 1015550, autotaxin inhibitors such as GLPG-1690 or BBT-877; connective tissue growth factor (CTGF) blocking antibodies such as Pamrevlumab; B-cell activating factor receptor (BAFF-R) blocking antibodies such as Lanalumab, alpha-V/beta-6 blocking inhibitors such as BG-00011/STX100, recombinant pentraxin-2 (PTX-2) such as PRM-151; c-Jun-N-terminal kinase (JNK) inhibitors such as CC-90001; galectin-3 inhibitors such as TD-139; G-protein coupled receptor 84 (GPR84) inhibitors; G-protein coupled receptor 84/G-protein coupled receptor 40 dual inhibitors such asPBI-4050, Rho Associated Coiled-Coil Containing Protein Kinase 2 (ROCK2) inhibitors such as KD-025, heat shock protein 47 (HSP47) small interfering RNA such as BMS-986263/ND-L02-s0201; Wnt pathway inhibitor such as SM-04646; LD4/PDE3/4 inhibitors such as Tipelukast; recombinant immuno-modulatory domains of histidyl tRNA synthetase(HARS) such as ATYR-1923, prostaglandin synthase inhibitors such as ZL-2102/SAR-191801; 15-hydroxy-eicosapentaenoic acid (15-HEPE e.g. DS102); Lysyl Oxidase Like 2 (LOXL2) inhibitors such as PAT-1251, PXS-5382/PXS-5338; phosphoinositide 3-kinases (PI3K)/mammalian target of rapamycin (mTOR) dual inhibitors such as HEC-68498; calpain inhibitors such as BLD-2660; mitogen-activated protein kinase kinase kinase (MAP3K19) inhibitors such as MG-S-2525; chitinase inhibitors such as OATD-01,mitogen-activated protein kinase-activated protein kinase 2 (MAPKAPK2) inhibitors such as MMI-0100; transforming growth factor beta I (TGF-beta I) small interfering RNA such as TRKZSO/BNC-1021; or lysophosphatidic acid receptor antagonists such as BMS986278.

The dosage for the combination partners mentioned above is usually 1/5 of the lowest dose normally recommended up to 1/1 of the normally recommended dose.

Therefore, in another aspect, this invention relates to the use of a compound according to the invention in combination with one or more additional therapeutic agents described hereinbefore and hereinafter for the treatment of diseases or conditions which may be affected or which are mediated by QPCT/L, in particular diseases or conditions as described hereinbefore and hereinafter.

In a further aspect this invention relates to a method for treating a disease or condition which can be influenced by the inhibition of QPCT/L in a patient that includes the step of administering to the patient in need of such treatment a therapeutically effective amount of a compound of formula (I) or a pharmaceutically acceptable salt thereof in combination with a therapeutically effective amount of one or more additional therapeutic agents.

In a further aspect this invention relates to the use of a compound of formula (I) or a pharmaceutically acceptable salt thereof in combination with one or more additional therapeutic agents for the treatment of diseases or conditions which can be influenced by the inhibition of QPCT/L in a patient in need thereof.

In yet another aspect the present invention relates to a method for the treatment of a disease or condition mediated by QPCT/L activity in a patient that includes the step of administering to the patient, preferably a human, in need of such treatment a therapeutically effective amount of a compound of the present invention in combination with a therapeutically effective amount of one or more additional therapeutic agents described in hereinbefore and hereinafter.

The use of the compound according to the invention in combination with the additional therapeutic agent may take place simultaneously or at staggered times.

The compound according to the invention and the one or more additional therapeutic agents may both be present together in one formulation, for example a tablet or capsule, or separately in two identical or different formulations, for example as a so-called kit-of-parts.

Consequently, in another aspect, this invention relates to a pharmaceutical composition that comprises a compound according to the invention and one or more additional therapeutic agents described hereinbefore and hereinafter, optionally together with one or more inert carriers and/or diluents.

Other features and advantages of the present invention will become apparent from the following more detailed examples which illustrate, by way of example, the principles of the invention.

### Preparation

The compounds according to the present invention and their intermediates may be obtained using methods of synthesis which are known to the one skilled in the art and described in the literature of organic synthesis. Preferably, the compounds are obtained in analogous fashion to the methods of preparation explained more fully hereinafter, in particular as described in the experimental section. In some cases, the order in carrying out the reaction steps may be varied. Variants of the reaction methods that are known to the one skilled in the art but not described in detail here may also be used.

The general processes for preparing the compounds according to the invention will become apparent to the one skilled in the art studying the following schemes. Any functional groups in the starting materials or intermediates may be protected using conventional protecting groups. These protecting groups may be cleaved again at a suitable stage within the reaction sequence using methods familiar to the one skilled in the art.

The compounds according to the invention are prepared by the methods of synthesis described hereinafter in which the substituents of the general formulae have the meanings given herein before. These methods are intended as an illustration of the invention without restricting its subject matter and the scope of the compounds claimed to these examples. Where the preparation of starting compounds is not described, they are commercially obtainable or may be prepared analogously to known compounds or methods described herein. Substances described in the literature are prepared according to the published methods of synthesis. Abbreviations are as defined in the Examples section.

Example 1 may be prepared as shown in Scheme I below.

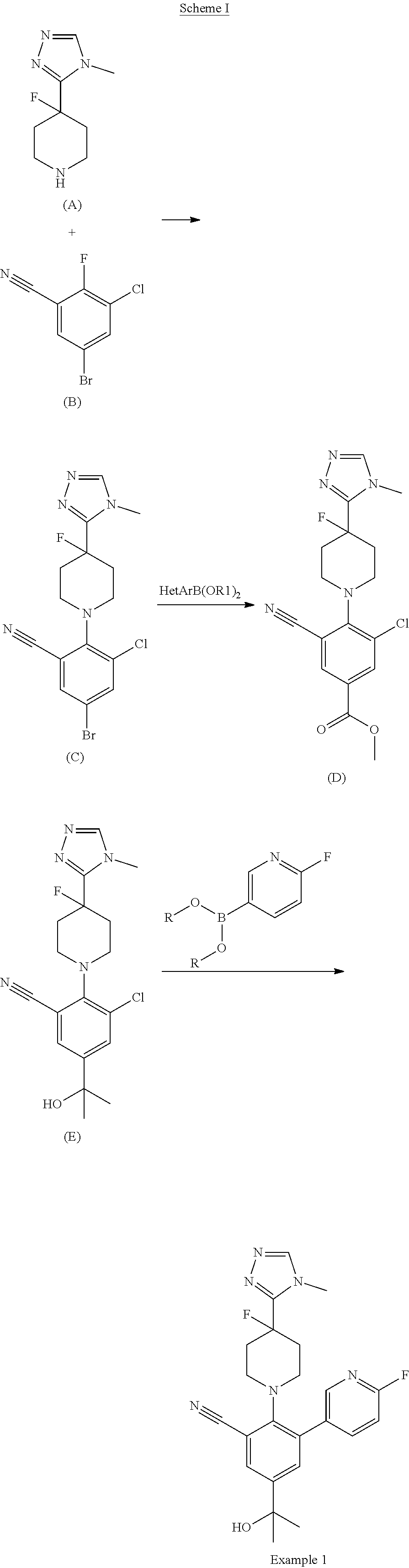

Compound (C) can be prepared by reaction of piperidines (A) with fluoro-benzonitriles (B) in the presence of a suitable base (e.g. diisopropylethylamine). The aryl bromide moiety can be transformed into the corresponding ester upon reaction with a suitable catalyst (e.g. Pd(OAc)2+dppf) under a CO atmosphere in the presence of a suitable alcohol (e.g. methanol). Compound (D) may be further converted into tertiary alcohol (E) upon reaction with a suitable organometallic reagent (e.g. MeMgBr). The intermediate (E) is then subjected to a Suzuki-cross coupling with a fluoro-pyridine boronic acid derivative in the presence of a suitable catalyst (e.g. Pd XPhos G4) and a suitable base at elevated temperature (e.g. 100° C.) to afford example 1.

Examples 2-9 may be prepared as shown in Scheme II below.

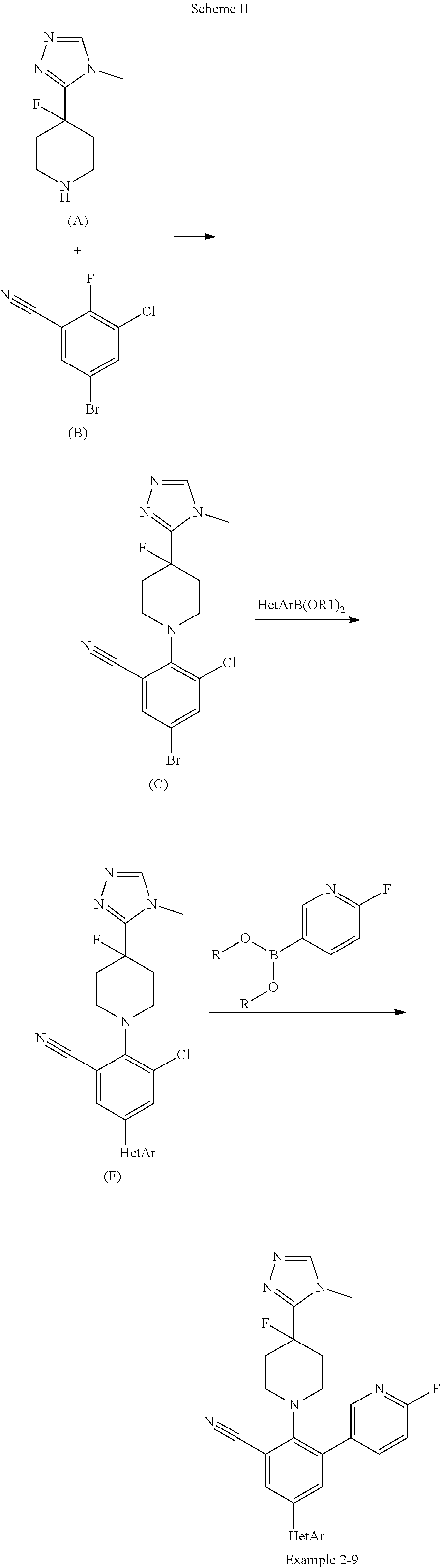

Compounds (C) can be prepared by reaction of piperidines (A) with fluoro-benzonitriles (B) in the presence of a suitable base (e.g. di-isopropylethylamine). The reaction can typically be run at ambient temperature or at elevated temperature (up to 110° C.) in the presence of a base (e.g. diisopropylethylamine). The intermediate (C) is then subjected to a Suzuki-cross coupling with a hetero-aryl boronic acid derivative in the presence of a suitable catalyst (e.g. Pd(dtbpf)Cl₂) and a suitable base at elevated temperature (e.g. 100° C.) to afford compounds of general formula (F). Examples 2-9 can be obtained by Suzuki-cross coupling with a fluoro-pyridine boronic acid derivative in the presence of a suitable catalyst (e.g. Pd XPhos G3) and a suitable base at elevated temperature (e.g. 100° C.) to afford examples 2-9.

Compounds of formula (A) may be prepared as shown in Scheme III below:

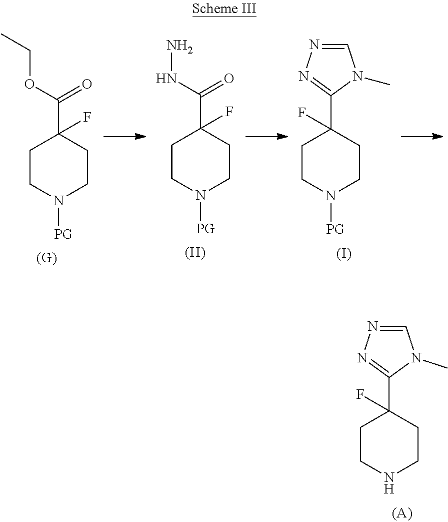

Compounds of formula (A) can be prepared from the corresponding piperidinyl esters (G) equipped with a suitable protecting group (PG, e.g. BOC) by treatment with a suitable hydrazine source (e.g. N₂H₄*H₂O) at elevated temperature (e.g. 50° C.). The obtained hydrazide (H) is then activated with DMF/DMA at elevated temperature (e.g. 50° C.) and subsequently treated with methyl amine at elevated temperature (e.g. 90° C.) to yield the triazole derivative (I). Compounds of formula (A) can be obtained by cleaving the protecting group under suitable conditions (e.g. TFA).

### EXAMPLES

### Preparation

The compounds according to the invention and their intermediates may be obtained using methods of synthesis which are known to the one skilled in the art and described in the literature of organic synthesis for example using methods described in “Comprehensive Organic Transformations”, 2nd Edition, Richard C. Larock, John Wiley & Sons, 2010, and “March's Advanced Organic Chemistry”, 7th Edition, Michael B. Smith, John Wiley & Sons, 2013. Preferably the compounds are obtained analogously to the methods of preparation explained more fully hereinafter, in particular as described in the experimental section. In some cases the sequence adopted in carrying out the reaction schemes may be varied. Variants of these reactions that are known to the skilled artisan but are not described in detail herein may also be used. The general processes for preparing the compounds according to the invention will become apparent to the skilled man on studying the schemes that follow. Starting compounds are commercially available or may be prepared by methods that are described in the literature or herein, or may be prepared in an analogous or similar manner. Before the reaction is carried out, any corresponding functional groups in the starting compounds may be protected using conventional protecting groups. These protecting groups may be cleaved again at a suitable stage within the reaction sequence using methods familiar to the skilled man and described in the literature for example in “Protecting Groups”, 3rd Edition, Philip J. Kocienski, Thieme, 2005, and “Protective Groups in Organic Synthesis”, 4th Edition, Peter G. M. Wuts, Theodora W. Greene, John Wiley & Sons, 2006. The terms “ambient temperature” and “room temperature” are used interchangeably and designate a temperature of about 20° C., e.g. between 19 and 24° C.

### Abbreviations

| | |
|---|---|
| ACN | acetonitrile |
| Aq. | aqueous |
| brine | saturated aqueous NaCl solution |
| ° C. | degree celsius |
| CyH/CH | cyclohexane |
| CO₂ | carbon dioxide |
| conc. | concentrated |
| Cs₂CO₃ | cesium carbonate |
| DCM | dichloromethane |
| DIPA | N,N-diisopropylamine |
| DIPEA | N,N-diisopropylethylamine |
| DMF | N,N-dimethylformamide |
| DMSO | dimethyl sulfoxide |
| ESI-MS | Electrospray ionisation mass spectrometry |
| EtOAc/EA | ethyl acetate |
| EtOH | ethanol |
| ex | example |
| eq | equivalent |
| FA | formic acid |
| h | hour |
| H₂O | water |
| HCl | hydrochloric acid |
| HPLC | high performance liquid chromatography |
| Int. | intermediate |
| K₂CO₃ | potassium carbonate |
| K₃PO₄ | tripotassium phosphate |
| KOAc | potassium acetate |
| KOH | potassium hydroxide |
| L | liter |
| LDA | lithium diisopropylamide |
| LiOH | lithium hydroxide |
| M | Molar (mol/L) |
| MeOH | methanol |
| MeTHF | methyl tetrahydrofuran |
| MgSO₄ | magnesium sulfate |
| min | minute |
| mL | milliliter |
| MTBE | Methyl-tert-butylether |
| μL | microliter |
| N₂ | nitrogen |
| n-BuLi | n-Butyllithium |
| NBS | N-Bromosuccinimide |
| NCS | N-Chlorosuccinimide |
| Na₂CO₃ | sodium carbonate |
| NaHCO₃ | sodium bicarbonate |
| NH₃ | ammonia |
| NH₄Cl | ammonium chloride |
| NaOH | sodium hydroxide |
| Na₂SO₄ | sodium sulfate |
| PdCl₂(PPh₃)₂ | Bis(triphenylphosphine)palladium(II) dichloride |
| Pd(dppf)Cl₂ | 1,1′-Bis(diphenylphosphino)ferrocene |
| | palladium(II)dichloride |
| Pd(dtbpf)Cl₂ | 1,1′-Bis(di-tert-butylphosphino)ferrocene |
| | palladium(II)dichloride |
| Pd(PPh₃)₄ | Tetrakis(triphenylphosphine)palladium(0) |
| PE | petroleum ether |
| Prep. | preparative |
| RP | reversed phase |
| RT/rt | room temperature (about 20° C.) |
| sat. | saturated |
| SFC | Supercritical Fluid Chromatography |
| SiO₂ | silica |
| TEA | triethylamine |
| TFA | trifluoroacetic acid |
| TFAA | trifluoroacetic anhydride |
| THF | tetrahydrofuran |
| Xphos Pd G3 | (2-Dicyclohexylphosphino-2′,4′,6′-triisopropyl- |
| | 1,1′-biphenyl)[2-(2′-amino-1,1′- |
| | biphenyl)]palladium(II) methanesulfonate |
| XPhos Pd G4 | ((SP-4-3)-[Dicyclohexyl[2′,4′,6′- |
| | tris(1-methylethyl)[1,1′-bipheny]]-2- |
| | yl]phosphine](methanesulfonato-κO)[2′- |
| | (methylamino-κN)[1,1′-bi-phenyl]-2-yl- |
| | κC]palladium |

### PREPARATION OF INTERMEDIATES

### Synthesis of Intermediate I.1

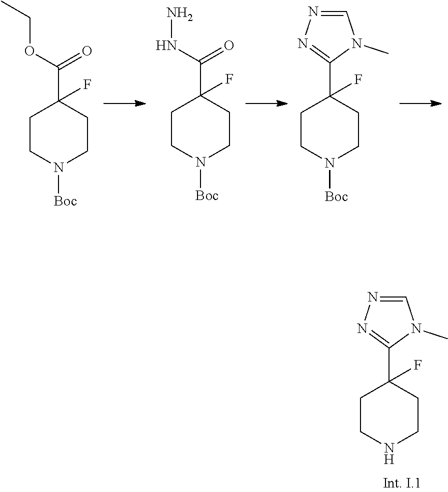 [SMILES: CCOC(=O)C1(F)CCN(C(=O)OC(C)(C)C)CC1 ; CC(C)(C)OC(=O)N1CCC(F)(C(=O)NN)CC1 ; Cn1cnnc1C1(F)CCN(C(=O)OC(C)(C)C)CC1 ; Cn1cnnc1C1(F)CCNCC1]

### tert-Butyl 4-fluoro-4-(hydrazinecarbonyl)piperidine-1-carboxylate

1-tert-Butyl 4-ethyl 4-fluoropiperidine-1,4-dicarboxylate (160 g, 0.58 mol) is suspended in ethanol (640 mL) in a round-bottom flask. Hydrazine hydrate (70.6 mL, 1.16 mol) is added to the mixture at ambient temperature. The reaction mixture is heated to 50° C. and stirred for 12 h. After cooling to ambient temperature, the mixture is concentrated under reduced pressure to yield tert-butyl 4-fluoro-4-(hydrazinecarbonyl)piperidine-1-carboxylate in 80% purity.
C₁₁H₂₀FN₃O₃(M=261.3 g/mol)
ESI-MS: 284 [M+Na]⁺
Rt (HPLC): 0.62 min (method A)

### tert-Butyl 4-fluoro-4-(4-methyl-4H-1,2,4-triazol-3-yl)piperidine-1-carboxylate

tert-Butyl 4-fluoro-4-(hydrazinecarbonyl)piperidine-1-carboxylate (135 g, 0.413 mol, 80% purity) is mixed with dioxane (945 mL) in a round-bottom-flask. N,N-Dimethylformamiddimethylacetal (137 mL, 1.03 mol) is added to the mixture at ambient temperature. The reaction mixture is heated to 50° C. and stirred for 1 h. A solution of methylamine (299 g, 30% in EtOH, 2.89 mol) and acetic acid (165 mL, 2.89 mol) are added into the mixture. The resulting reaction mixture is heated to 90° C. and stirred for 11 h. After cooling to ambient temperature, the mixture is concentrated under reduced pressure. The residue is purified by column chromatography (SiO₂, PE/EtOAc gradient 20:1 to 0:1) to obtain tert-butyl 4-fluoro4-(4-methyl-4H-1,2,4-triazol-3-yl)piperidine-1-carboxylate.
C₁₃H₂₁FN₄O₂(M=284.3 g/mol)
ESI-MS: 285 [M+H]⁺
Rt (HPLC): 0.77 min (method A)

### Intermediate I.1: 4-fluoro-4-(4-methyl-4H-1,2,4-triazol-3-yl)piperidine

tert-Butyl 4-fluoro-4-(4-methyl-4H-1,2,4-triazol-3-yl)piperidine-1-carboxylate (90 g, 0.32 mol) is combined with methanol (90 mL) in a round-bottom flask. A solution of HCl (4 m in MeOH, 450 mL, 1.8 mol) is added slowly at ambient temperature. The resulting reaction mixture is stirred at ambient temperature for 12 h. The desired product is collected by filtration, washed with methanol, and dried to yield 4-fluoro-4-(4-methyl-4H-1,2,4-triazol-3-yl)piperidine hydrochloride salt.

The hydrochloride salt (13.5 g) is added to a solution of ammonia in methanol (7 M, 150 mL) and purified by column chromatography (Biotage SNAP Cartridge KP-NH 110 g, gradient DCM/MeOH 4:1 to 7:3) to afford the title compound.
C₈H₁₃FN₄ (M=184.2 g/mol)
ESI-MS: 185 [M+H]⁺
Rt (HPLC): 0.20 min (method B)

### Intermediate II.1: 5-bromo-3-chloro-2-fluorobenzonitrile

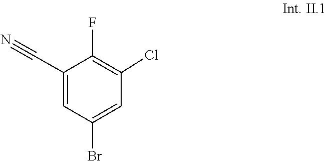 [SMILES: N#Cc1cc(Br)cc(Cl)c1F]

Int. II.1 (5-bromo-3-chloro-2-fluorobenzonitrile) was obtained from commercial suppliers (CAS: 1000577-76-3).

### Intermediate III.1: 5-bromo-3-chloro-2-[4-fluoro-4-(4-methyl-4H1,2,4-triazol-3-yl)piperidin-1-yl]benzonitrile

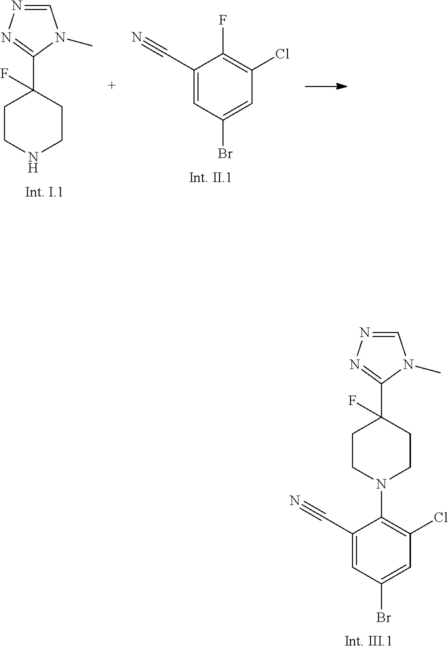

Int. II.1 (1.5 g, 6.21 mmol) is suspended in a mixture of DMSO (10.0 mL) and DIPEA (2.1 mL, 12.4 mmol) and Int. I.1 (1.5 g, 6.21 mmol) is added. The resulting reaction mixture is stirred at 50° C. for 86 h. The reaction mixture is cooled to 5° C. before water (5 mL) is added and the mixture is stirred for another 30 min. The precipitated solid is collected by filtration, washed with water and dried to afford the desired compound.
C₁₅H₄BrClFN₅ (M=398.7 g/mol)
ESI-MS: 398/400 [M+H]⁺
Rt (HPLC): 0.69 min (method B)

### Synthesis of Intermediate V.1: 3-chloro-2-[4-fluoro-4-(4-methyl-4H-1,2,4-triazol-3-yl)piperidin-1-yl]-5-(2-hydroxypropan-2-yl)benzonitrile

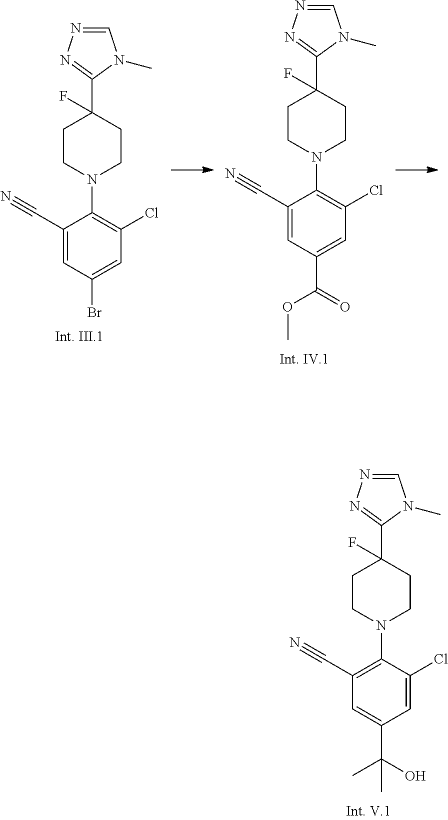 [SMILES: Cn1cnnc1C1(F)CCN(c2c(Cl)cc(Br)cc2C#N)CC1 ; COC(=O)c1cc(Cl)c(N2CCC(F)(c3nncn3C)CC2)c(C#N)c1 ; Cn1cnnc1C1(F)CCN(c2c(Cl)cc(C(C)(C)O)cc2C#N)CC1]

### Intermediate IV.1

To a mixture of Int. III.1 (2.8 g, 7.02 mmol) in MeOH (10 mL) and 1,4-dioxane (10 mL) in a high-pressure reaction vessel is added Pd(OAc)2 (306 mg, 1.36 mmol), 1,1′-bis(diphenylphosphino)ferrocene (389 mg, 0.702 mmol) and sodium acetate (1.53 g, 18.6 mmol). A CO pressure of 8 bar is applied and the reaction mixture stirred at 60° C. for 24 h. After cooling to ambient temperature and releasing the pressure, the mixture is concentrated, suspended in DMF and purified by preparative HPLC (Zorbax StableBond C18, MeCN/water gradient containing 0.3% TFA).
C₁₇H₁₇C1FN₅O₂(M=377.8 g/mol)
ESI-MS: 378 [M+H]⁺
Rt (HPLC): 0.86 min (method C)

### Intermediate V.1

To a mixture of Int. IV.1 (50 mg, 132 μmol) in THE (4.0 mL) is added a solution of methylmagnesium bromide (3.2 M in THF, 0.097 mL, 291 μmol) at 0° C. under vigorous stirring. The reaction mixture is warmed to ambient temperature and stirred for 2 h. The mixture is concentrated and the residue purified by column chromatography (SiO2, EtOAc/MeOH gradient) to yield the title compound.
C₁₈H₂₁ClFN₅O (M=377.8 g/mol)
ESI-MS: 378 [M+H]⁺
Rt (HPLC): 0.79 min (method D)

### Synthesis of Intermediate V.2: 3-chloro-2-[4-fluoro-4-(4-methyl-4H-1,2,4-triazol-3-yl)piperidin-1-yl]-5-(2-methyl-1,3-oxazol-5-yl)benzonitrile

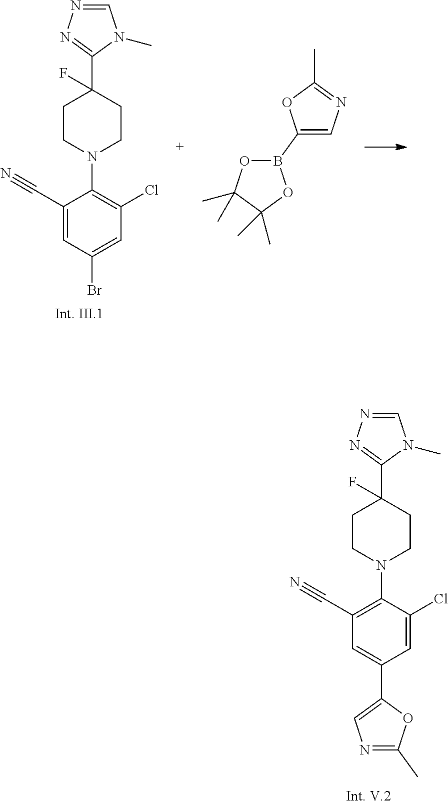 [SMILES: Cn1cnnc1C1(F)CCN(c2c(Cl)cc(Br)cc2C#N)CC1 ; Cc1ncc(B2OC(C)(C)C(C)(C)O2)o1 ; Cc1ncc(-c2cc(Cl)c(N3CCC(F)(c4nncn4C)CC3)c(C#N)c2)o1]

To a mixture of Int. III.1 (100 mg, 251 μmol), 2-methyl-5-(4,4,5,5-tetramethyl-1,3,2-dioxaborolan-2-yl)oxazole (66.2 mg, 301 μmol) in 1,4-dioxane (3.0 mL) is added a solution of potassium carbonate (2 M in water, 376 μL, 753 μmol). The resulting mixture is purged by passing an argon stream through the mixture for 5 min. Pd(dtbpf)Cl₂ (16.3 mg, 25.1 μmol) is added, and the mixture is further purged for 2 min. The reaction mixture is stirred at 85° C. for 18 h. After cooling to ambient temperature, the mixture is diluted with acetonitrile and filtered. The filtrate is purified by preparative HPLC (XBridge C18 column, ACN/water gradient containing 0.1% NH₃) to yield the title compound.
C₁₉H₁₈ClFN₆O (M=400.8 g/mol)
ESI-MS: 401 [M+H]⁺
Rt (HPLC): 0.84 min (method E)

### Intermediates Synthesized Analogous to the Procedure Described for Int. V.1

| | | | | Molecular For- |
|---|---|---|---|---|
| | | | | mula (MW) |
| | | | | ESI-MS |
| | Starting | | Deviation from | HPLC retention |
| Int. | material | Structure | general procedure | time (method) |
| V.3 | Int. III.1 + (1,3,5-tri- methyl-1H- pyrazol-4- yl)boronic acid | 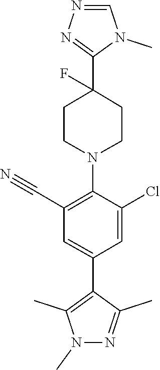 [SMILES: Cc1nn(C)c(C)c1-c1cc(Cl)c(N2CCC(F)(c3nncn3C)CC2)c(C#N)c1] | — | C₂₁H₂₃ClFN₇ (M = 427.9 g/mol) ESI-MS: 428 [M + H]⁺ Rt (HPLC): 0.85 min (method E) |
| V.4 | Int. III.1 + 1,3- dimethyl-4- (4,4,5,5- tetramethyl- 1,3,2- dioxaborolan-2- yl)-1H- pyrazole | 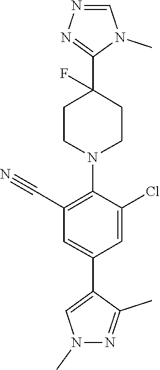 [SMILES: Cc1nn(C)cc1-c1cc(Cl)c(N2CCC(F)(c3nncn3C)CC2)c(C#N)c1] | — | C₂₀H₂₁ClFN₇ (M = 413.9 g/mol) ESI-MS: 414 [M + H]⁺ Rt (HPLC): 0.60 min (method B) |
| V.5 | Int. III.1 + 1-Methyl- 1H pyrazole- 4- boronic acid | 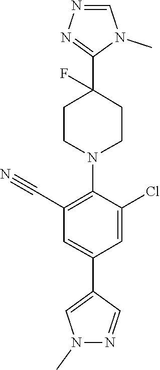 [SMILES: Cn1cc(-c2cc(Cl)c(N3CCC(F)(c4nncn4C)CC3)c(C#N)c2)cn1] | Pd(dtbpf)C12 and boronic acid were added again after 18 h at 80° C.; stirred again for 18 h at 80° C. purification by preparative HPLC (Sunfire C18, MeCN/water gradient containing 0.1% TFA) | C₁₉H₁₉ClFN₇ (M = 399.9 g/mol) ESI-MS: 400 [M + H]⁺ Rt (HPLC): 0.69 min (method F) |
| V.6 | Int. III.1 + 3-(4,4,5,5- tetramethyl- 1,3,2- dioxaboro- lan-2- yl)pyridine | 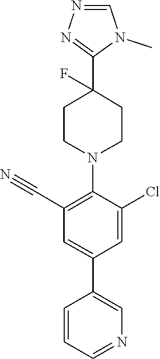 [SMILES: Cn1cnnc1C1(F)CCN(c2c(Cl)cc(-c3cccnc3)cc2C#N)CC1] | Purified by pre- parative HPLC (Sunfire C18, MeCN/water gradient containing 0.1% TFA) | C₂₀H₁₈ClFN₆ (M = 396.9 g/mol) ESI-MS: 397 [M + H]⁺ Rt (HPLC): 0.52 min (method F) |
| V.7 | Int. III.1 + (2- methyl- pyrimidin-5- yl)boronic acid | 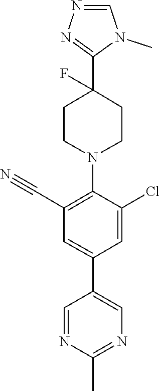 [SMILES: Cc1ncc(-c2cc(Cl)c(N3CCC(F)(c4nncn4C)CC3)c(C#N)c2)cn1] | — | C₂₀H₁₉ClFN₇ (M = 411.9 g/mol) ESI-MS: 412 [M + H]⁺ Rt (HPLC): 0.79 min (method E) |
| V.8 | Int. III.1 + 4-(4,4,5,5- tetra- methyl-1,3,2- dioxaboro- lan-2- yl)pyridazine | 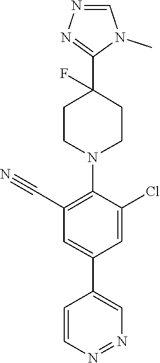 [SMILES: Cn1cnnc1C1(F)CCN(c2c(Cl)cc(-c3ccnnc3)cc2C#N)CC1] | — | C₁₉H₁₇ClFN₇ (M = 397.8 g/mol) ESI-MS: 398 [M + H]⁺ Rt (HPLC): 0.73 min (method E) |
| V.9 | Int. III. 1 + (2-(trifluoro- methyl) pyrimidin-5- yl)boronic acid | 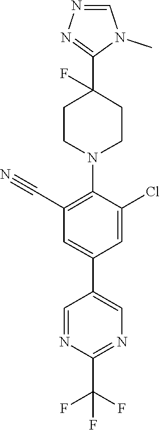 [SMILES: Cn1cnnc1C1(F)CCN(c2c(Cl)cc(-c3cnc(C(F)(F)F)nc3)cc2C#N)CC1] | Purified by pre- parative HPLC (Sunfire C18, MeCN/water containing 0.1% TFA) | C₂₀H₁₆ClF₄N₇ (M = 465.8 g/mol) ESI-MS: 466 [M + H]⁺ Rt (HPLC): 0.88 min (method F) |

### Preparation of Final Compounds

### Example 1

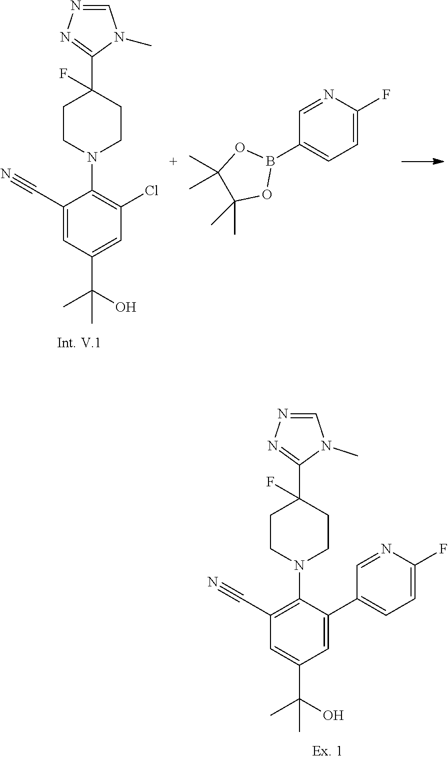

To a mixture of Int. V.1 (35.0 mg, 92.6 μmol) and 2-fluoro-5-(4,4,5,5-tetramethyl-1,3,2-dioxaborolan-2-yl)pyridine (32.3 mg, 0.139 mmol) in 1,4-dioxane (1 mL) is added a solution of potassium carbonate (2 M in water, 139 μL, 0.278 mmol). The resulting mixture is purged by passing an argon stream through the mixture. XPhos Pd G4 ((SP-4-3)-[Dicyclohexyl[2′,4′,6′-tris(1-methylethyl)[1,1′-biphenyl]-2-yl]phosphine](methanesulfonato-KO)[2′-(methylamino-xN)[1,1′-biphenyl]-2-yl-κC]palladium, CAS: 1599466-81-5) (4.1 mg, 4.6 μmol) is added, and the mixture is further purged with argon. The reaction mixture is stirred at 100° C. for 5 h. After cooling to ambient temperature, the mixture is concentrated and purified by column chromatography (SiO₂, EtOAc/MeOH gradient) to yield the title compound.

### Example 2

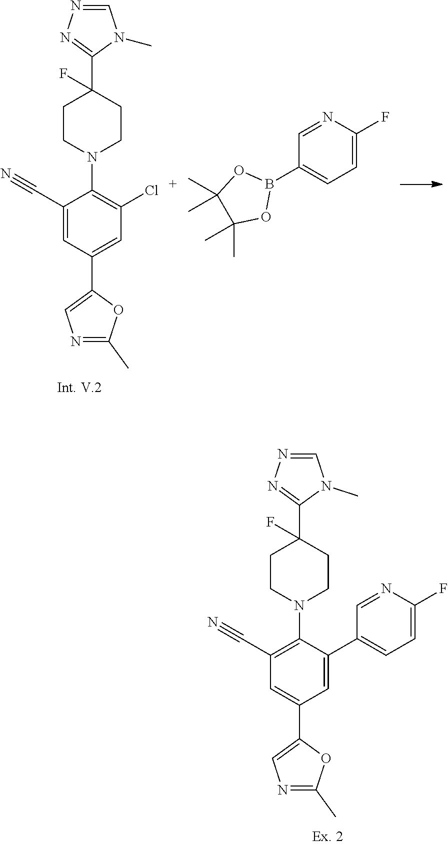

To a mixture of Int. V.2 (26.0 mg, 64.8 μmol) and 2-fluoro-5-(4,4,5,5-tetramethyl-1,3,2-dioxaborolan-2-yl)pyridine (20.7 mg, 90.8 μmol) in 1,4-dioxane (1.5 mL) is added a solution of sodium carbonate (2 M in water, 81.1 μL, 0.162 mmol). The resulting mixture is purged by passing an argon stream through the mixture. XPhos Pd G3 ((2-Dicyclohexylphosphino2′,4′,6′-triisopropyl-1,1′-biphenyl)[2-(2′-amino-1,1′-biphenyl)]palladium(II) methanesulfonate, CAS: 1445085-55-1) (4.1 mg, 4.9 μmol) is added, and the mixture is further purged with argon for 5 min. The reaction mixture is stirred at 95° C. for 12 h. After cooling to ambient temperature, the mixture is diluted with an ACN/water mixture, filtered, and purified by preparative HPLC (Sunfire C18 column, ACN/water gradient containing 0.1% TFA) to yield the title compound.

### Examples Synthesized Analogous to the Procedure Described for Example 2

| | | | Deviation from general |
|---|---|---|---|
| example | Starting materials | Structure | procedure |
| 3 | V.3 + 2-fluoro-5-(4,4,5,5- tetramethyl-1,3,2- dioxaborolan-2-yl) pyridine | 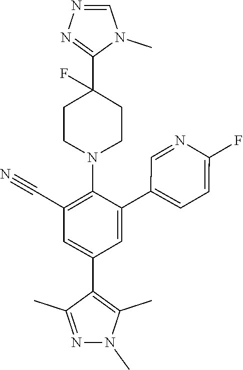 [SMILES: Cc1nn(C)c(C)c1-c1cc(C#N)c(N2CCC(F)(c3nncn3C)CC2)c(-c2ccc(F)nc2)c1] | — |
| 4 | V.4 + 2-fluoro-5-(4,4,5,5- tetramethyl-1,3,2- dioxaborolan-2-yl) pyridine | 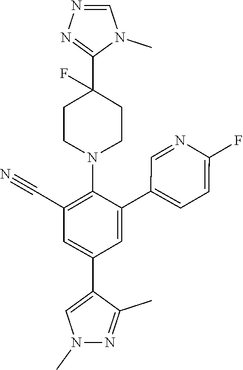 [SMILES: Cc1nn(C)cc1-c1cc(C#N)c(N2CCC(F)(c3nncn3C)CC2)c(-c2ccc(F)nc2)c1] | 4 h at 95° C.; Purification by preparative HPLC (X-Bridge C18, MeCN/water gradient containing 0.1% NH3) |
| 5 | V.5 + 2-fluoro-5-(4,4,5,5- tetramethyl-1,3,2- dioxaborolan-2-yl) pyridine | 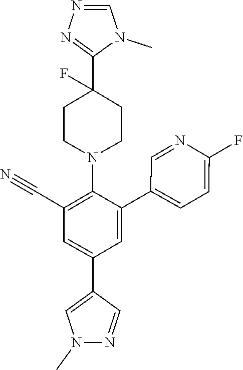 [SMILES: Cn1cc(-c2cc(C#N)c(N3CCC(F)(c4nncn4C)CC3)c(-c3ccc(F)nc3)c2)cn1] | 4 h at 95° C.; Purification by preparative HPLC (X-Bridge C18, MeCN/water gradient containing 0.1% NH3) |
| 6 | V.6 + 2-fluoro-5-(4,4,5,5- tetramethyl-1,3,2- dioxaborolan-2-yl) pyridine | 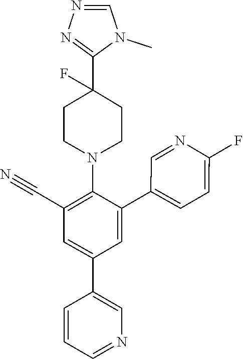 [SMILES: Cn1cnnc1C1(F)CCN(c2c(C#N)cc(-c3cccnc3)cc2-c2ccc(F)nc2)CC1] | 3.5 eq. Na₂CO₃ solution |
| 7 | V.7 + 2-fluoro-5-(4,4,5,5- tetramethyl-1,3,2- dioxaborolan-2-yl) pyridine | 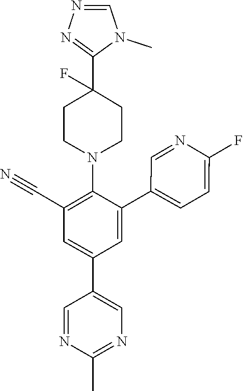 [SMILES: Cc1ncc(-c2cc(C#N)c(N3CCC(F)(c4nncn4C)CC3)c(-c3ccc(F)nc3)c2)cn1] | 3.5 eq. Na₂CO₃ solution; 22 h at 95° C. |
| 8 | V.8 + 2-fluoro-5-(4,4,5,5- tetramethyl-1,3,2- dioxaborolan-2-yl) pyridine | 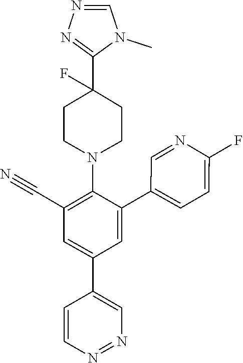 [SMILES: Cn1cnnc1C1(F)CCN(c2c(C#N)cc(-c3ccnnc3)cc2-c2ccc(F)nc2)CC1] | 3.5 eq. Na₂CO₃ solution; 22 h at 95° C. |
| 9 | V.9 + 2-fluoro-5-(4,4,5,5- tetramethyl-1,3,2- dioxaborolan-2-yl) pyridine | 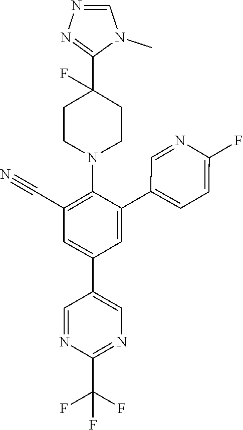 [SMILES: Cn1cnnc1C1(F)CCN(c2c(C#N)cc(-c3cnc(C(F)(F)F)nc3)cc2-c2ccc(F)nc2)CC1] | 3.5 eq. Na₂CO₃ solution; 22 h at 95° C. |

### Analytical Data of Synthesized Examples

| | | Molecular Formula | |
|---|---|---|---|
| | | (MW) | |
| | | ESI-MS | |
| | | HPLC retention time | ¹H NMR (400 MHz, |
| example | Structure | (method) | DMSO-d6): δ in ppm |
| 1 | 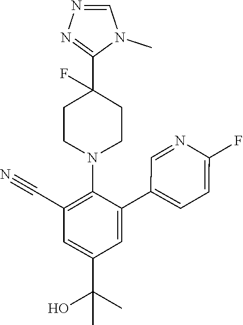 [SMILES: Cn1cnnc1C1(F)CCN(c2c(C#N)cc(C(C)(C)O)cc2-c2ccc(F)nc2)CC1] | C₂₃H₂₄F₂N₆O (M = 438.5 g/mol) ESI-MS: 439 [M + H]⁺ Rt (HPLC): 0.81 min (method D) | 8.47 (s, 1 H), 8.30 (d, J = 2.4 Hz, 1 H), 8.08 (td, J = 8.2, 2.5 Hz, 1 H), 7.83 (d, J = 2.3 Hz, 1 H), 7.62 (d, J = 2.3 Hz, 1 H), 7.30 (dd, J = 8.4, 2.7 Hz, 1 H), 5.23 (s, 1 H), 3.72 (d, J = 1.5 Hz, 3 H), 3.13- 3.26 (m, 2 H), 3.04- 3.12 (m, 2 H), 2.00- 2.21 (m, 4 H), 1.45 (s, 6 H) |
| 2 | 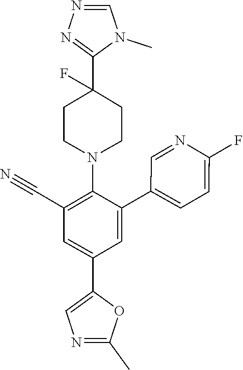 [SMILES: Cc1ncc(-c2cc(C#N)c(N3CCC(F)(c4nncn4C)CC3)c(-c3ccc(F)nc3)c2)o1] | C₂₄H₂₁F₂N₇O (M = 461.5 g/mol) ESI-MS: 462 [M + H]⁺ Rt (HPLC): 0.69 min (method G) | 8.56 (s, 1 H), 8.36 (d, J = 2.5 Hz, 1 H), 8.13 (td, J = 8.2, 2.5 Hz, 1 H), 8.09 (d, J = 2.2 Hz, 1 H), 7.82 (d, J = 2.2 Hz, 1 H), 7.65 (s, 1 H), 7.34 (dd, J = 8.5, 2.7 Hz, 1 H), 3.73 (d, J = 1.5 Hz, 3 H), 3.10-3.25 (m, 4 H), 2.47 (s, 3 H), 2.02-2.22 (m, 4 H) |
| 3 | 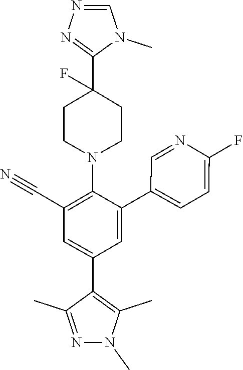 [SMILES: Cc1nn(C)c(C)c1-c1cc(C#N)c(N2CCC(F)(c3nncn3C)CC2)c(-c2ccc(F)nc2)c1] | C₂₆H₂₆F₂N₈ (M = 488.5 g/mol) ESI-MS: 489 [M + H]⁺ Rt (HPLC): 0.65 min (method G) | 8.62 (s, 1 H), 8.34 (d, J = 2.4 Hz, 1 H), 8.12 (td, J = 8.2, 2.5 Hz, 1 H), 7.68 (d, J = 2.2 Hz, 1 H), 7.42 (d, J = 2.2 Hz, 1 H), 7.31 (dd, J = 8.4, 2.6 Hz, 1 H), 3.75 (d, J = 1.6 Hz, 3 H), 3.70 (s, 3 H), 3.09- 3.28 (m, 4 H), 2.25 (s, 3 H), 2.05-2.21 (m, 7 H) |
| 4 | 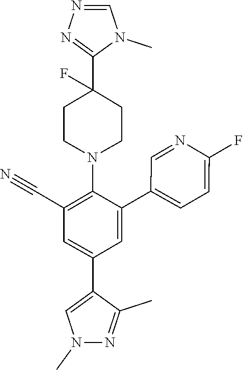 [SMILES: Cc1nn(C)cc1-c1cc(C#N)c(N2CCC(F)(c3nncn3C)CC2)c(-c2ccc(F)nc2)c1] | C₂₅H₂₄F₂N₈ (M = 474.5 g/mol) ESI-MS: 475 [M + H]⁺ Rt (HPLC): 0.58 min (method B) | 8.48 (s, 1 H), 8.36 (d, J = 2.4 Hz, 1 H), 8.14 (td, J = 8.2, 2.5 Hz, 1 H), 8.04 (s, 1 H), 7.82 (d, J = 2.3 Hz, 1 H), 7.60 (d, J = 2.3 Hz, 1 H), 7.32 (dd, J = 8.5, 2.7 Hz, 1 H), 3.78 (s, 3 H), 3.73 (d, J = 1.6 Hz, 3 H), 3.15- 3.27 (m, 2 H), 3.07- 3.14 (m, 2 H), 2.32 (s, 3 H), 2.01-2.21 (m, 4 H) |
| 5 | 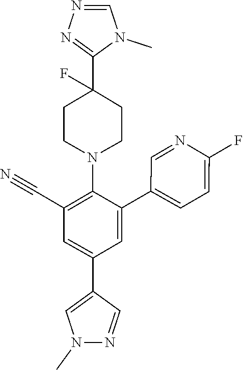 [SMILES: Cn1cc(-c2cc(C#N)c(N3CCC(F)(c4nncn4C)CC3)c(-c3ccc(F)nc3)c2)cn1] | C₂₄H₂₂F₂N₈ (M = 460.5 g/mol) ESI-MS: 461 [M + H]⁺ Rt (HPLC): 0.57 min (method B) | 8.48 (s, 1 H), 8.36 (d, J = 2.4 Hz, 1 H), 8.28 (s, 1 H), 8.13 (td, J = 8.2, 2.5 Hz, 1 H), 8.04 (d, J = 2.3 Hz, 1 H), 8.00 (d, J = 0.6 Hz, 1 H), 7.80 (d, J = 2.2 Hz, 1 H), 7.33 (dd, J = 8.4, 2.6 Hz, 1 H), 3.86 (s, 3 H), 3.73 (d, J = 1.6 Hz, 3 H), 3.15- 3.26 (m, 2 H), 3.05- 3.14 (m, 2 H), 1.99- 2.20 (m, 4 H) |
| 6 | 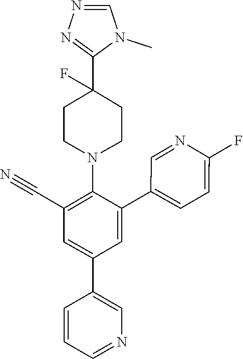 [SMILES: Cn1cnnc1C1(F)CCN(c2c(C#N)cc(-c3cccnc3)cc2-c2ccc(F)nc2)CC1] | C₂₅H₂₁F₂N₇ (M = 457.5 g/mol) ESI-MS: 458 [M + H]⁺ Rt (HPLC): 0.47 min (method G) | 9.18 (d, J = 2.2 Hz, 1 H), 8.74 (dd, J = 5.1, 1.2 Hz, 1 H), 8.55-8.60 (m, 2 H), 8.43 (d, J = 2.4 Hz, 1 H), 8.32 (d, J = 2.3 Hz, 1 H), 8.18 (td, J = 8.2, 2.5 Hz, 1 H), 8.03 (d, J = 2.3 Hz, 1 H), 7.79 (dd, J = 8.1, 5.2 Hz, 1 H), 7.36 (dd, J = 8.5, 2.7 Hz, 1 H), 3.75 (d, J = 1.5 Hz, 3 H), 3.14-3.28 (m, 4 H), 2.07-2.27 (m, 4 H) |
| 7 | 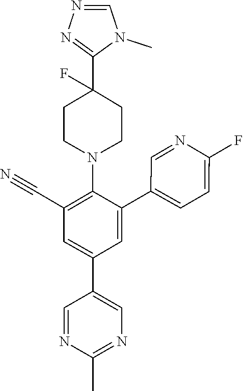 [SMILES: Cc1ncc(-c2cc(C#N)c(N3CCC(F)(c4nncn4C)CC3)c(-c3ccc(F)nc3)c2)cn1] | C₂₅H₂₂F₂N₈ (M = 472.5 g/mol) ESI-MS: 473 [M + H]⁺ Rt (HPLC): 0.46 min (method H) | 9.14 (s, 2 H), 8.60 (s, 1 H), 8.41 (d, J = 2.4 Hz, 1 H), 8.31 (d, J = 2.4 Hz, 1 H), 8.17 (td, J = 8.2, 2.5 Hz, 1 H), 8.02 (d, J = 2.3 Hz, 1 H), 7.35 (dd, J = 8.5, 2.7 Hz, 1 H), 3.75 (d, J = 1.5 Hz, 3 H), 3.12-3.28 (m, 4 H), 2.67 (s, 3 H), 2.05-2.24 (m, 4 H) |
| 8 | 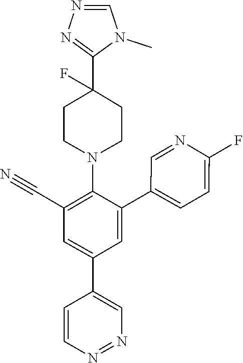 [SMILES: Cn1cnnc1C1(F)CCN(c2c(C#N)cc(-c3ccnnc3)cc2-c2ccc(F)nc2)CC1] | C₂₄H₂₀F₂N₈ (M = 458.5 g/mol) ESI-MS: 459 [M + H]⁺ Rt (HPLC): 0.40 min (method H) | 9.76 (dd, J = 2.5, 1.1 Hz, 1 H), 9.29 (dd, J = 5.6, 1.1 Hz, 1 H), 8.48 (s, 1 H), 8.47 (d, J = 2.4 Hz, 1 H), 8.43 (d, J = 2.4 Hz, 1 H), 8.18 (td, J = 8.3, 2.6 Hz, 1 H), 8.13-8.15 (m, 2 H), 7.36 (dd, J = 8.5, 2.7 Hz, 1 H), 3.73 (d, J = 1.6 Hz, 3 H), 3.16- 3.25 (m, 4 H), 2.08- 2.25 (m, 4 H) |
| 9 | 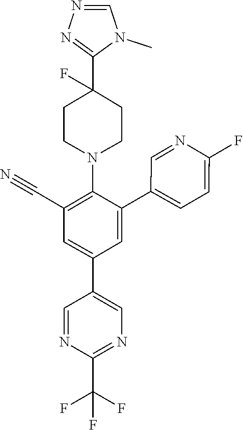 [SMILES: Cn1cnnc1C1(F)CCN(c2c(C#N)cc(-c3cnc(C(F)(F)F)nc3)cc2-c2ccc(F)nc2)CC1] | C₂₅H₁₉F₅N₈ (M = 526.5 g/mol) ESI-MS: 527 [M + H]⁺ Rt (HPLC): 0.59 min (method H) | 9.51 (s, 2 H), 8.54 (s, 1 H), 8.45 (d, J = 2.3 Hz, 1 H), 8.43 (d, J = 2.4 Hz, 1 H), 8.18 (td, J = 8.2, 2.5 Hz, 1 H), 8.15 (d, J = 2.4 Hz, 1 H), 7.37 (dd, J = 8.4, 2.6 Hz, 1 H), 3.74 (d, J = 1.5 Hz, 3 H), 3.12-3.31 (m, 4 H), 2.08-2.26 (m, 4 H) |

### Analytical HPLC Methods

Method A

| time | Vol % water | Vol % ACN | Flow |
|---|---|---|---|
| (min) | (incl. 0.04% TFA) | (incl. 0.02% TFA) | [mL/min] |
| 0.00 | 95 | 5 | 1.5 |
| 0.70 | 5 | 95 | 1.5 |
| 1.16 | 5 | 95 | 1.5 |
| 1.50 | 95 | 5 | 1.5 |

Analytical column: Kinetex EVO C18_2.1 × 30 mm_5 μm; column temperature: 40° C.

Method B

| time | Vol % water | | Flow |
|---|---|---|---|
| (min) | (incl. 0.1% NH₃) | Vol % ACN | [mL/min] |
| 0.00 | 95 | 5 | 1.3 |
| 0.02 | 95 | 5 | 1.3 |
| 1.00 | 0 | 100 | 1.3 |
| 1.30 | 0 | 100 | 1.3 |

Device description: Waters Acquity; Analytical column: XBridge (Waters) BEH C18_2.1 × 30 mm_2.5 μm; column temperature: 60° C.

Method C

| time | Vol % water | | Flow |
|---|---|---|---|
| (min) | (incl. 0.1% TFA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Zorbax (Agilent) StableBond C18_3.0 × 30 mm_1.8 μm; column temperature: 60° C.

Method D

| time | Vol % water | | Flow |
|---|---|---|---|
| (min) | (incl. 0.1% FA) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Sunfire C18_3.0 × 30 mm_2.5 μm; column temperature: 60° C.

Method E

| time | Vol % water | | Flow |
|---|---|---|---|
| (min) | (incl. 0.1% NH₃) | Vol % ACN | [mL/min] |
| 0.00 | 97 | 3 | 2.2 |
| 0.20 | 97 | 3 | 2.2 |
| 1.20 | 0 | 100 | 2.2 |
| 1.25 | 0 | 100 | 3.0 |
| 1.40 | 0 | 100 | 3.0 |

Device description: Agilent 1200; Analytical column: Xbridge (Waters) C18_3.0 × 30 mm_2.5 μm; column temperature: 60° C.

Method F

| time | Vol % water | | Flow |
|---|---|---|---|
| (min) | (incl. 0.1% TFA) | Vol % ACN | [mL/min] |
| 0.00 | 95 | 5 | 1.5 |
| 1.30 | 0 | 100 | 1.5 |
| 1.50 | 0 | 100 | 1.5 |

Device description: Waters Acquity; Analytical column: Sunfire (Waters) C18_3.0 × 30 mm_2.5 μm; column temperature: 60° C.

Method G

| time | Vol % water | Vol % ACN | Flow |
|---|---|---|---|
| (min) | (incl. 0.1% TFA) | (incl. 0.08% TFA) | [mL/min] |
| 0.00 | 95 | 5 | 1.5 |
| 1.30 | 0 | 100 | 1.5 |
| 1.50 | 0 | 100 | 1.5 |
| 1.60 | 95 | 5 | 1.5 |

Device description: Waters Acquity; Analytical column: Sunfire (Waters) C18_3.0 × 30 mm_2.5 μm; column temperature: 60° C.

Method H

| time | Vol % water | | Flow |
|---|---|---|---|
| (min) | (incl. 0.1% TFA) | Vol % ACN | [mL/min] |
| 0.00 | 99 | 1 | 1.6 |
| 0.02 | 99 | 1 | 1.6 |
| 1.00 | 0 | 100 | 1.6 |
| 1.10 | 0 | 100 | 1.6 |

Device description: Waters Acquity; Analytical column: Xbridge (Waters) BEH C18_2.1 × 30 mm_1.7 μm; column temperature: 60° C.

1. A compound of formula (I) 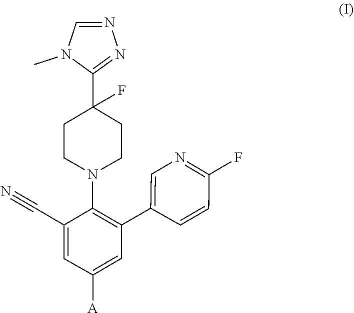
wherein
A is HOC(CH₃)₂C, 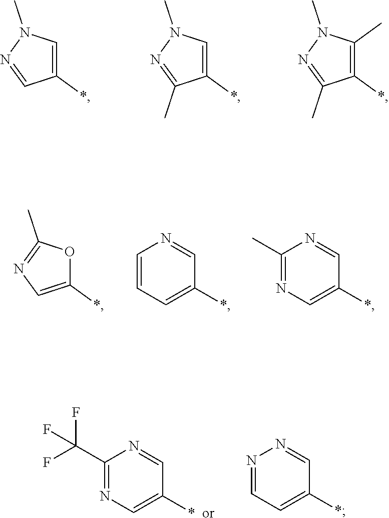
or a salt thereof, particularly a pharmaceutically acceptable salt thereof.

2. The compound of formula (I) according to claim 1, selected from the group consisting of 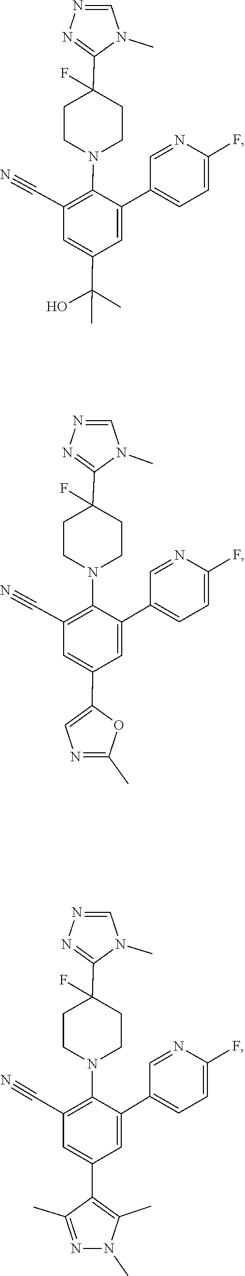 [SMILES: Cn1cnnc1C1(F)CCN(c2c(C#N)cc(C(C)(C)O)cc2-c2ccc(F)nc2)CC1 ; Cc1ncc(-c2cc(C#N)c(N3CCC(F)(c4nncn4C)CC3)c(-c3ccc(F)nc3)c2)o1 ; Cc1nn(C)c(C)c1-c1cc(C#N)c(N2CCC(F)(c3nncn3C)CC2)c(-c2ccc(F)nc2)c1] 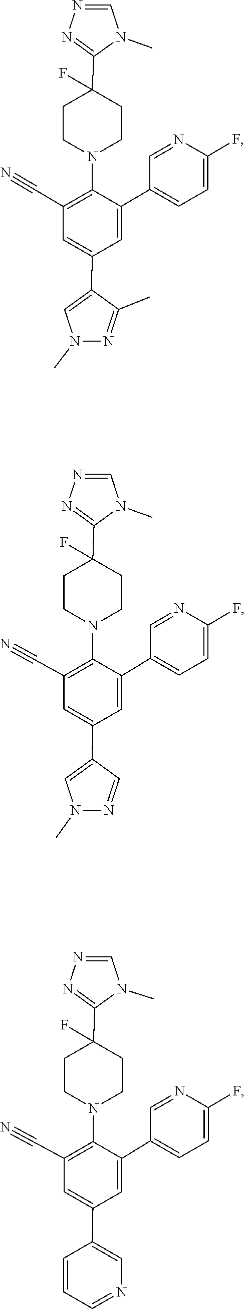 [SMILES: Cc1nn(C)cc1-c1cc(C#N)c(N2CCC(F)(c3nncn3C)CC2)c(-c2ccc(F)nc2)c1 ; Cn1cc(-c2cc(C#N)c(N3CCC(F)(c4nncn4C)CC3)c(-c3ccc(F)nc3)c2)cn1 ; Cn1cnnc1C1(F)CCN(c2c(C#N)cc(-c3cccnc3)cc2-c2ccc(F)nc2)CC1] 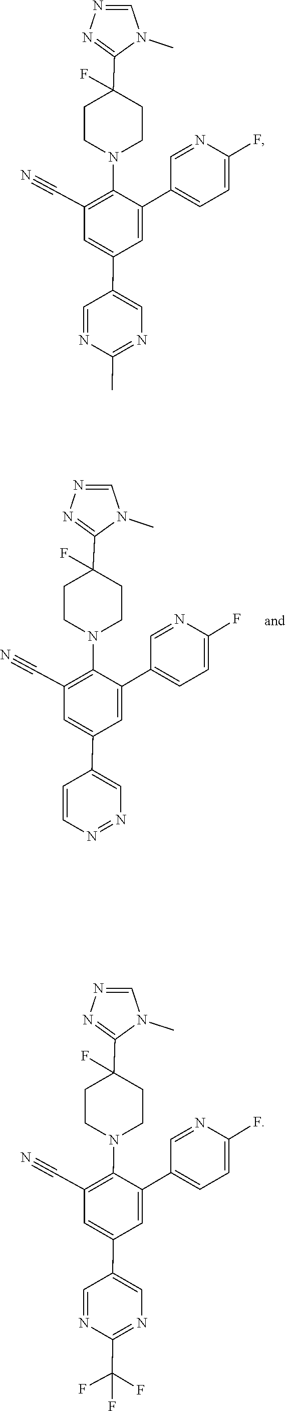 [SMILES: Cc1ncc(-c2cc(C#N)c(N3CCC(F)(c4nncn4C)CC3)c(-c3ccc(F)nc3)c2)cn1 ; Cn1cnnc1C1(F)CCN(c2c(C#N)cc(-c3ccnnc3)cc2-c2ccc(F)nc2)CC1 ; Cn1cnnc1C1(F)CCN(c2c(C#N)cc(-c3cnc(C(F)(F)F)nc3)cc2-c2ccc(F)nc2)CC1]
or a salt thereof.

3. A pharmaceutically acceptable salt of a compound according to claim 1.

4. A pharmaceutical composition comprising one or more compounds according to claim 1, or pharmaceutically acceptable salts thereof, optionally together with one or more inert carriers and/or diluents.

5. A pharmaceutical composition comprising one or more compounds according to claim 1, or pharmaceutically acceptable salts thereof, and one or more additional therapeutic agents, together with one or more inert carriers and/or diluents.

6. The pharmaceutical composition according to claim 5 wherein the one or more additional therapeutic agents are selected from the group consisting of anticancer agents and antifibrotic agents.

7. (canceled)

8. A method for the treatment of a disease in a patient in need thereof, the comprising administering one or more compounds according to claim 1 or pharmaceutically acceptable salts thereof to the patient.

9. The method according to claim 8 wherein the disease is selected from the group consisting of cancer, fibrotic diseases, neurodegenerative diseases, atherosclerosis, infectious diseases, and chronic kidney diseases.
